(19) United States
(12) Patent Application Publication
(10) Pub. No.: US 2006/0258000 A1
(43) Pub. Date: Nov. 16, 2006

(54) Use of steady-state oxygen gradients to modulate animal cell functions

(76) Inventors: Jared Whiting Allen, Los Angeles, CA (US); Sangeeta N. Bhatia, Lexington, MA (US)

Correspondence Address: BUCHANAN, INGERSOLL & ROONEY LLP, P.O. BOX 1404, ALEXANDRIA, VA, 22313-1404, US

(21) Appl. No.: 10/547,057

(22) PCT Filed: Feb. 26, 2004
(86) PCT No.: PCT/US04/06018

§ 371 Date: Jun. 15, 2006

Related U.S. Application Data
(60) Provisional application No. 60/450,532, filed on Feb. 26, 2003.

(51) Int. Cl. C12N 5/06; C12M 1/00; C12N 5/08

(52) U.S. Cl. 435/325; 435/289.1; 435/370

(57) ABSTRACT

The disclosure provides a bioreactor that allows steady-state oxygen gradients to be imposed upon in vitro culture systems. The bioreactor system of the disclosure has been applied to liver zonation and have shown that physiological oxygen gradients contribute to heterogeneity of tissue cultures in vitro.

### CROSS REFERENCE TO RELATED APPLICATIONS

This application claims priority under 35 U.S.C. §119 from Provisional Application Ser. No. 60/450,532, filed Feb. 26, 2003, the disclosure of which is incorporated herein by reference.

### STATEMENT AS TO FEDERALLY SPONSORED RESEARCH

The U.S. Government has certain rights in this invention pursuant to Grant No. DK56966 awarded by the National Institutes of Health.

### TECHNICAL FIELD

The disclosure relates to methods and apparati for culturing tissue. More particularly, the disclosure relates to bioreactors capable of growing and sustaining liver cells in a diffusion gradient bioreactor system.

### BACKGROUND

Cell culture techniques and understanding of the complex interactions cells have with one another and the surrounding environment have improved in the past decade. There is now a better understanding of the role extracellular matrix materials play in the proliferation and development of artificial tissues in vitro. Historically cell culture techniques and tissue development fail to take into account the necessary microenvironment for cell-cell and cell-matrix communication as well as an adequate diffusional environment for delivery of nutrients and removal of waste products.

While many methods and bioreactors have been developed to grow tissue masses for the purposes of generating artificial tissues for transplantation or for toxicology studies, these bioreactors do not adequately simulate in vitro the mechanisms by which nutrients and gases are delivered to tissue cells in vivo. For example, cells in living tissue are “polarized” with respect to diffusion gradients. Differential delivery of oxygen and nutrients, as occurs in vivo by means of the capillary system, controls the relative functions of tissue cells and perhaps their maturation as well. Thus, bioreactors that do not simulate these in vivo delivery mechanisms do not provide a sufficient corrolary to in vivo environments to develop tissues or measure tissue responses in vitro.

The ability to develop in vitro tissue, such as hepatic tissue, can provide a supply of tissue for toxicology testing, extracorporeal liver devices as well as tissue for transplantation. For example, liver failure is the cause of death of over 30,000 patients in the United States every year and over 2 million patients worldwide. Current treatments are largely palliative—including delivery of fluids and serum proteins. The only therapy proven to alter mortality is orthotopic liver transplants; however, organs are in scarce supply (McGuire et al., Dig Dis. 13(6):379-88 (1995)).

Cell-based therapies have been proposed as an alternative to whole organ transplantation, a temporary bridge to transplantation, and/or an adjunct to traditional therapies during liver recovery. Three main approaches have been proposed: transplantation of isolated hepatocytes via injection into the blood stream, development and implantation of hepatocellular tissue constructs, and perfusion of blood through an extracorporeal circuit containing hepatocytes. Investigation in all three areas has dramatically increased in the last decade, yet progress has been stymied by the propensity for isolated hepatocytes to rapidly lose many key liver-specific functions.

### SUMMARY

Provided is a method comprising controlling an oxygen gradient across a population of cells in one or more bioreactors to modify a tissue morphology, function, and/or gene expression. In one aspect, the bioreactor comprises a pump, a gas exchange device, at least one culture device comprising, at least one housing; at least one substrate, at least one tissue binding surface on each of the at least one substrate, wherein the housing comprises at least one wall, an inlet port and an outlet port, wherein the housing fluidly seals the tissue binding surface to provide a flow space in fluid communication with the inlet and outlet ports, a gas sensor, and a fluid reservoir, wherein the pump, the gas exchange device, the culture device, the gas sensor and the fluid reservoir are in fluid communication, such that a fluid is pumped from the fluid reservoir through (i) the gas exchanger, (ii) the culture device, (iii) the gas sensor and returned to the fluid reservoir using the pump and wherein the population of cells is cultured on the tissue binding surface of the substrate and wherein the gas concentration is modulated by the gas exchange device and sensed by the gas sensor.

The disclosure further provides a bioreactor comprising: at least one housing having an inlet port and an outlet port; at least one substrate disposed in the at least one housing; at least one tissue binding surface on each of the at least one substrate, the housing and tissue binding surface defining a flow space along the tissue binding surface; a pump in fluid communication with the inlet port and the outlet port of the housing; a gas exchange device disposed between the pump and the inlet port; a fluid reservoir in fluid communication with the pump; and a gas sensor disposed between the outlet port and the fluid reservoir, wherein the pump, the gas exchange device, the flow space, the gas sensor and the fluid reservoir are in fluid communication, such that a fluid is pumped from the fluid reservoir through (i) the gas exchanger, (ii) the flow space, (iii) the gas sensor and returned to the fluid reservoir using the pump and wherein the gas concentration is modulated by the gas exchange device and sensed by the gas sensor.

Also provided is a method of producing a tissue, comprising: seeding a population of cells on a substrate in a bioreactor system; controlling an oxygen gradient across the population of cells in one or more bioreactors; culturing the cells under conditions and for a sufficient period of time to generate a tissue.

The disclosure futher provides a tissue produced by the methods and the bioreactors of the disclosure.

An assay system is also provided. The assay system comprising: contacting a tissue produced by the method disclosed herein with a test agent and measuring an activity selected from gene expression, cell function, metabolic activity, morphology, and a combination thereof, of the tissue.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1 is a schematic depicting blood flow in the liver and zonation along the sinusoid.

FIG. 2 depicts examples of bioreactor systems.

FIG. 3 is a schematic showing a bioreactor system of the disclosure.

FIG. 4 is a schematic of a high-throughput, micro-bioreactor array. Bottom panel depicts array of 50 micro-bioreactors in ten modules of 5 micro-bioreactors each. Modules are laid out on a 4-inch glass wafer with 2 alignment holes. Reactors are formed by an underlying glass surface that is micropatterned with collagen and a silicone “lid” that confines the flow of perfusate. Each module has a single inlet and single outlet. Middle panel depicts 3 of the 5 micro-bioreactors in a module with a common inlet and outlet. Top panel depicts micropatterned co-cultures with aligned hepatocytes and fibroblasts in each micro-bioreactor.

FIG. 5 shows a two-dimensional contour plot of predicted oxygen concentration profile in bioreactor cross-section. Output shows oxygen distribution with inlet pO₂ of 158-mmHg and flow rate 0.35 mL/min (Re=0.3) using the experimental parameters listed in Table 1.

FIG. 6A-B show oxygen transport models. (A) Flow rate dependence of bioreactor oxygen gradients. Model output for flow rate ranging from 0.5 to 2 mL/min with a fixed inlet pO₂ of 76 mmHg is shown for both the analytical and numerical solutions to Eq. (1). (B) Inlet pO₂ dependence of bioreactor oxygen gradients. With a fixed flow rate of 0.5 mL/min, the effect of various inlet pO₂ from 75 to 175 mmHg is shown from the analytical solution. Regions of oxygen tension that correspond to a typical periportal and perivenous O₂ levels are depicted.

FIG. 7 is a plot showing experimental validation of oxygen transport. Measured outlet oxygen concentration was measured as a function of flow rate at inlet pO₂ of 76 and 158 mmHg and compared to predicted values. Both the analytical and numerical model predictions are represented. Data points represent the mean and SEM of three separate experiments.

FIG. 8A-B are photos of cells that show validation of hypoxic cellular environment at the bioreactor outlet. A bioreactor of the disclosure was operated at 0.3 mL/min with inlet pO₂ of 76 mmHg. Higher intensity stain in outlet region (B) over the inlet (A) indicates the presence of a local hypoxic environment.

FIG. 9A-F are photos showing morphology and viability of cells in a bioreactor system of the disclosure. Representative phase-contrast micrographs (A, C, E) from three regions of the bioreactor used for morphology analysis. Fluorescence images (B, D, F) indicating culture viability, reported as the mean±SEM from three distinct image fields. Images were acquired after 24-h perfusion at 0.35 mL/min with 158-mmHg inlet pO₂. Changes in viability along the length of the chamber were not statistically significant (P<0.05).

FIG. 10A-C shows protein induction by oxygen gradients in a bioreactor. Heterogeneous induction of PEPCK and CYP2B by oxygen gradients. Bioreactors were operated with an inlet pO₂ of 76 and 158 mmHg and flow rate of 0.5 mL/min. The resulting cell surface oxygen gradients are shown schematically as calculated from the numerical model (A). Western blots of PEPCK (B) and CYP2B (C) protein levels from four regions along the bioreactor substrate were analyzed to determine relative optical density. In both cases, when the bioreactor was operated with physiologic gradient (low inlet), a heterogeneous induction was observed, whereas imposing a supraphysiologic gradient (control, high inlet) resulted in a more uniform protein distribution. Blots were processed in separate experiments, enabling only qualitative comparison between conditions. Normalization of band densities to the maximal density from both experiments is meant to facilitate comparison.

FIG. 11A-B shows a western blot analysis performed on cell lysates obtain from 4 separate regions along the length of the bioreactor. (A) Protein levels of CYP2B and CYP3A from static culture and 36-hour perfused cultures without chemical induction were compared. (B) Similar analysis was performed on 36-hour bioreactor cultures containing the indicated levels of PB, DEX, or EGF. (C) Shows data related to CYP2B and CYP3A in hepatocyte only cultures and co-cultures.

FIG. 12 shows the viability of co-cultures and hepatocyte-only cultures as assessed by MTT after 24-hour exposure to varying concentrations of APAP.

FIG. 13 shows photomicrograph of cultures stained with MTT after 24 hours perfusion with indicated concentrations of APAP.

FIG. 14 shows relative viability of co-cultures perfused with APAP. Bright field images of MTT stained, perfused cultures were acquired from 5 regions along the length of the slide. Representative images from 15 mM APAP treatment are shown. The mean optical density of each image was determined and normalized to control cultures. Mean and SEM from 3 fields in each region is depicted. Values were normalized to controls and represent the mean and SEM of 3 cultures.

Like reference symbols in the various drawings indicate like elements.

### DETAILED DESCRIPTION

The disclosure provides a bioreactor that allows steady-state oxygen gradients to be imposed upon in vitro culture systems. The bioreactor system of the disclosure has been applied to liver zonation and have shown that physiological oxygen gradients contribute to heterogeneous induction of PEPCK and CYP2B that mimics distributions in vivo. The results demonstrate the ability of oxygen to modulating gene expression and imply that oxygen plays an important role in the maintenance of liver-specific metabolism in a bioreactor system. In addition, considerations of the effect of oxygen gradients in the design and optimization current bioartificial support systems may serve to improve their function. Other applications of the gradient system might involve examination of ischemia-reperfusion injury, the mechanisms of ischemic preconditioning being attempted in organ preservation, and mechanisms of zonal toxicity such as that caused by carbon tetrachloride or acetaminophen. This approach is generally applicable to systems that can benefit from (i) a continuous range of O₂ concentration; (ii) dynamics; (iii) large cell populations for molecular characterization; and (iv) the role flow and soluble factors on cell function.

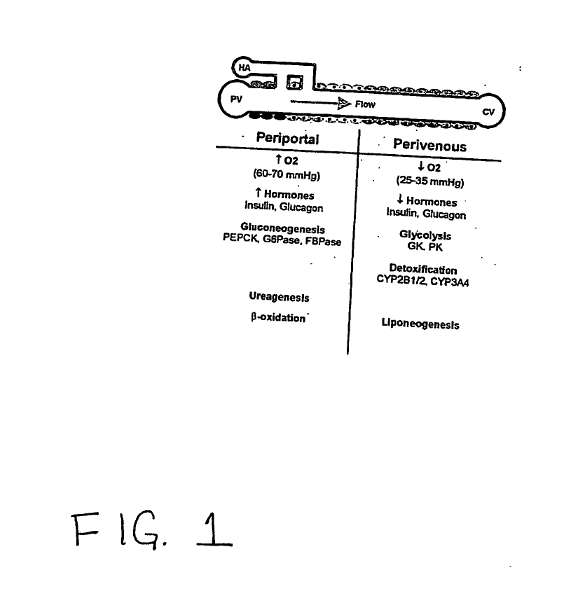

The morphology and function of cells in an organism vary with respect to their environment, including distance from sources of metabolites and oxygen. For example, the morphology and function of hepatocytes are known to vary with position along the liver sinusoids from the portal triad to the central vein (Bhatia et al., Cellular Engineering 1:125-135, 1996; Gebhardt R. Pharmaol Ther. 53(3):275-354, 1992; Jungermann K. Diabete Metab. 18(1):81-86, 1992; and Lindros, K. O. Gen Pharmacol. 28(2):191-6, 1997). This phenomenon, referred to a zonation, has been described in virtually all areas of liver function. Oxidative energy metabolism, carbohydrate metabolism, lipid metabolism, nitrogen metabolism, bile conjugation, and xenobiotic metabolism, have all been localized to separate zones (see, e.g., FIG. 1). Such compartmentalization of gene expression is thought to underlie the liver's ability to operate as a ‘glucostat’ as well as the pattern of zonal hepatotoxicity observed with some xenobiotics (e.g., environmental toxins, chemical/biological warfare agents, natural compounds such as holistic therapies and nutraceuticals).

In the liver, the periportal region surrounding the portal triad is rich in oxygen, hormones, and metabolic substrates drained from the gut. The perivenous region near the central vein is exposed to oxygen-depleted sinusoidal blood containing metabolic products secreted upstream. Key metabolic enzymes have been localized to each of these compartments. For example, the periportal region contains higher levels of phosphoenolpyruvate carboxykinase (PEPCK) and glucose-6-phosphatase (G6 Pase) essential for gluconeogenesis while the perivenous zone is relatively concentrated with glucokinase (GK) and pyruvate kinase (PK), rate-limiting enzymes of glycolysis. Several detoxification enzymes, such as CYP2B, CYP3A, and glucuronosyltransferase are also localized to the perivenous region. Zonation of cytochrome P450s along with a low oxygen environment are though to be the key mediators of perivenous toxicity of such compounds as carbon tetrachloride and acetaminophen.

Possible modulators of zonation include blood-borne hormones, O₂ tension, pH, extracellular matrix compositions, and innervations. The disclosure demonstrates that O₂ tension in vitro can be utilized to regulate lipid metabolism, urea synthesis, gluconeogenesis, and xenobiotic metabolism of isolated hepatocytes, thereby recreating different hepatocyte sub-populations based on the local O₂ concentration.

Disclosed herein is a bioreactor system that leverages the innate oxygen uptake process of mammalian cells to create a directional oxygen gradient in the perfusion reactor system. Directional oxygen gradients are present in various biological environments such as, for example, in cancer, tissue development, tissue regeneration, wound healing and in normal tissues. As a result of oxygen gradients along the length of a perfusion bioreactor system the cellular components exhibit different functional characteristics based on local oxygen availability.

The disclosure allows for the provision of controlled oxygen gradients over mammalian cells. Any perfusion bioreactor may experience the formation of oxygen and/or nutrient gradients to some degree but the bioreactor system disclosed herein is an express demonstration of imposing oxygen gradients to study and elicit specific cellular responses. Conventional cell culture systems exist in static oxygen environments and require multiple experiments and culture conditions to evaluate the effects of differential oxygen environments. The use of oxygen gradients in a perfusion system creates a continuum of oxygen concentrations over living cells. Existing bioreactor systems are almost exclusively designed and operated to prevent the formation of gradients along the length of a flow field or flow space. Furthermore, for many types of mammalian cells, a perfusion culture system provides a more favorable environment that is representative of the in viva environment. Such an approach offers the potential to study oxygen gradients in a manner similar to chemotactic soluble factors. Cellular responses that are otherwise unobserved may be uncovered by such a platform. Adaptation of the system to various cell types requires only aerobic metabolism and adhesion dependence of the cells.

The introduction of oxygen gradients in vitro has been applied to liver tissue using a flat plate bioreactor of the disclosure on which a monolayer of hepatocytes, either alone or in combination with other cells, is cultured. The operational parameters were controlled to predict and control oxygen gradients in the reactor. Co-culturing liver cells with other cell types in the reactor provides for long-term viability and differentiated liver function. The imposition of oxygen gradients in liver cell culture is significant because this condition mimics the gradients that are formed in the liver in viva. There are not other in vitro systems that mimic liver zonation in a flow-through platform amenable to interrogation with drug candidates.

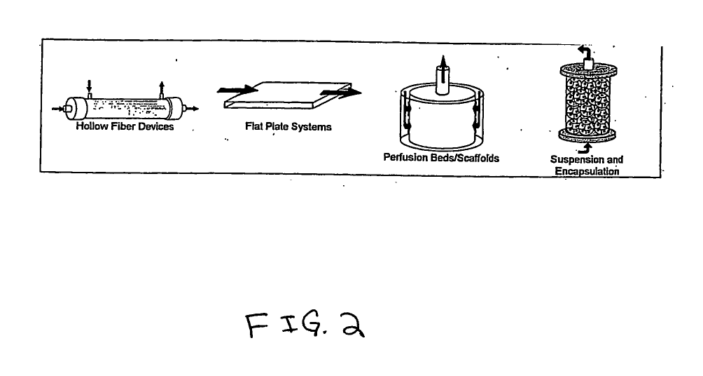

Several hepatocyte bioreactor designs have been developed that, to some extent, represent in vitro models of the liver. Such bioreactors can be classified at flat plate, hollow-fiber, packed bed, or perfused suspension (see, e.g., FIG. 2). A flat plate reactor provides a simple geometry, uniform cell distribution, and direct contact with perfusion media. When used in conjunction with sandwich cultures or co-cultures, flat plate reactors allow for a phenotypically-stable hepatocyte system for long-term studies. Hollow fiber designs adapted from the hemodialysis field have undergone extensive evaluation, although they are not designed to control the hepatocyte microenvironment. Reactors containing hepatocytes attached to microcarriers, seeded through microchanneled polyurethane, or embedded into woven scaffolds have also been proposed. As a rule, bioreactor platforms tend to be optimal either for (1) scale-up to clinical extracorporeal bioartificial liver devices (e.g., hollow fibers) or (2) highly controlled in vitro models of liver tissue for physiological and pathophysiological experimentation (e.g., flat plate reactors), but not both. One exception is a recently reported three-dimensional bioreactor that was developed based on the morphogenesis of hepatocytes into three-dimensional structures in an array of channels. While the design does allow for the perfusion of phenotypically-stable hepatocyte aggregates, it relies on tissue morphogenesis into a three-dimensional structure, which is inherently variable as compared to monolayer co-cultures that offer the advantage of a more reproducible transport interface. In addition, the reported design does not incorporate zonal variations or the ability to study multiple xenobiotics simultaneously. While the design allows for perfusion of phenotypically-stable hepatocyte aggregates, it relies on tissue morphogenesis of hepatocytes into a three-dimensional structure, which is inherently variable as compared to monolayer co-cultures that offer the advantage of a more reproducible transport interface. In addition, the foregoing design does not incorporate zonal variations or the ability to study multiple xenobiotics simultaneously.

The formation of oxygen gradients is achieved by optimizing parameters such as cell seeding density, flow rate, inlet oxygen concentration and reactor dimensions. Utilizing an in vitro system allows for molecular analysis of cellular responses including changes in gene expression, protein synthesis and cellular damage. The design principles of the oxygen gradient bioreactor are generally applicable to cell culture models in which oxygenation and oxygen availability affect cellular functions.

In one embodiment, the reactor can use primary hepatocytes as well as other cell types alone or in combination with hepatocytes (e.g., primary hepatocytes). Although, the examples provided herein utilize hepatocytes, other parenchymal and non-parenchymal cell types that can be used in the bioreactors and cultures systems of the disclosure include pancreatic cells (alpha, beta, gamma, delta), myocytes, enterocytes, renal epithelial cells and other kidney cells, brain cell (neurons, astrocytes, glia), respiratory epithelium, stem cells, and blood cells (e.g., erythrocytes and lymphocytes), adult and embryonic stem cells, blood-brain barrier cells, and other parenchymal cell types known in the art. The reactor can be used to culture parenchymal cells and stromal cells. For example, the reactor can be used with co-cultures of hepatocytes and stromal cells (e.g., fibroblasts). The scale of the reactor can be altered to allow for the fabrication of a high-throughput microreactor array to allow for interrogation of xenobiotics.

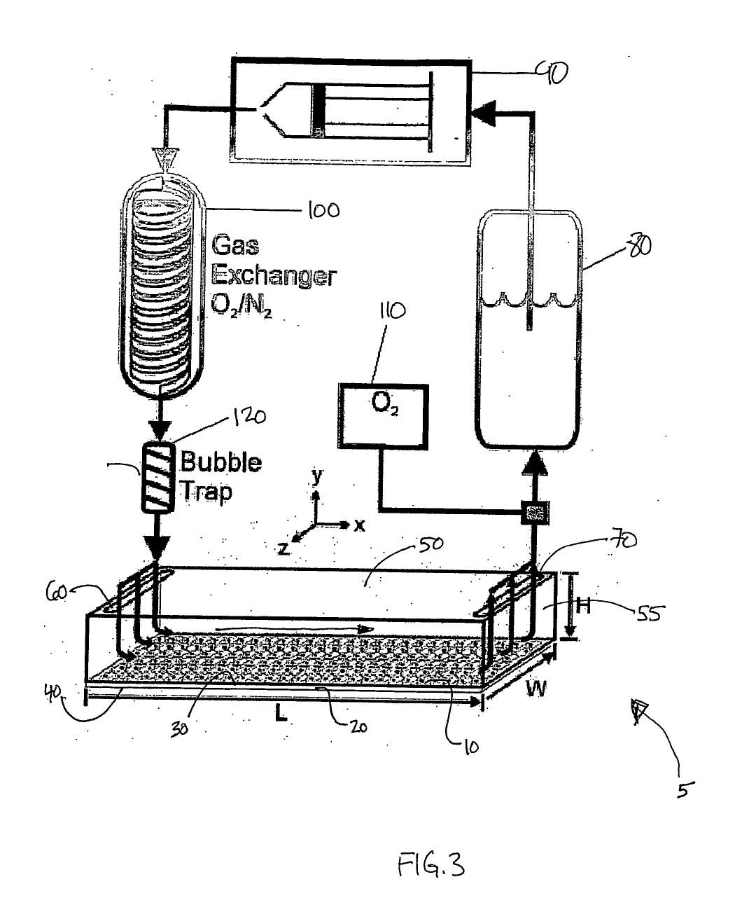

A bioreactor 5 of the disclosure (see, e.g., FIG. 3) comprises a pump 90, a gas exchange device 100, a bubble trap 120 a culture device 15 comprising a substrate 20, a tissue binding surface 30 and bottom surface 40, an enclosure/housing 50 having at least one wall 55, inlet port 60 and outlet port 70, O₂ sensor 110, and fluid reservoir 80. The bioreactor 5 comprises a pump 90 used to maintain circulation of fluid in the system. Pump 90 is in fluid communication with a gas exchange device 100 that oxygenates the fluid present in the system to a desired concentration. The pump 90 is also in fluid communication with fluid reservoir 80 used to contain, for example, nutrient media or other media to be contacted with cells in the system. In one aspect, the gas exchange device 100 is in fluid communication with a bubble trap 120 that serves to remove bubbles following gas exchange of the fluid in the gas exchange device 100. Fluid flowing through the system enters inlet port 60 of culture device 15 and passes across substrate 20 to outlet port 70. The inlet port 50 and outlet port 70 may be located on the x-, y-, or z-plane of the enclosure/housing 50.

In the specific embodiment of FIG. 3 the growth surface for cells is shown as being on top surface 30 of substrate 20, additional surfaces may be prepared for cell adherence and growth including any surface of housing/chamber 50 (i.e., any one or more walls chamber 50). In FIG. 3, cells are capable of growth on the top surface 30 of substrate 20. As discussed herein, the substrate 20 or one or more surfaces of housing/chamber 50 may be treated or modified to promote cellular adhesion to the substrate or improve cell growth. The cells may be grown in hydrogels and/or in porous or mesh materials present within the bioreactor system. Optical transparency of the substrate 20 and/or of the housing/chamber 50 is useful as a platform for conventional microscopy (fluorescent and transmitted light). Furthermore, in-line sensor can be incorporated using microtechnology. For example, molecular probes (e.g., probes that provide a measurable signal such as changes in fluorescence, electrical conductivity (including resistance, capacitance). Probes that can indicate a change include various green fluorescent protein molecules linked to various indicators that change conformation upon interacting with a molecule in the cellular milieu or media effluent. Probes that provide electrical changes upon interacting with a molecule in the cellular milieu or media effluent can include substrates that comprise various polymers (e.g. polypyrrole, polyaniline and the like, as well as semiconductive substrates) that have at least two conductive leads. Such substrates change resistance or capacitance upon interacting with a molecule. For example, each reactor (or a plurality of reactors in a microarray, as described herein) can have its own O₂, pH, metabolite sensor(s). Other sensor types are known in the art. In addition, methods of microfabrication for inclusion of such sensors are also known in the art.

Fluid, upon exiting culture device 15 through outlet port 70, contacts a gas sensor 110 (e.g., an oxygen sensor) that measures gas concentrations in the fluid. The data obtained from gas sensor 110 is used to modify gas exchange in the gas exchanger 100.

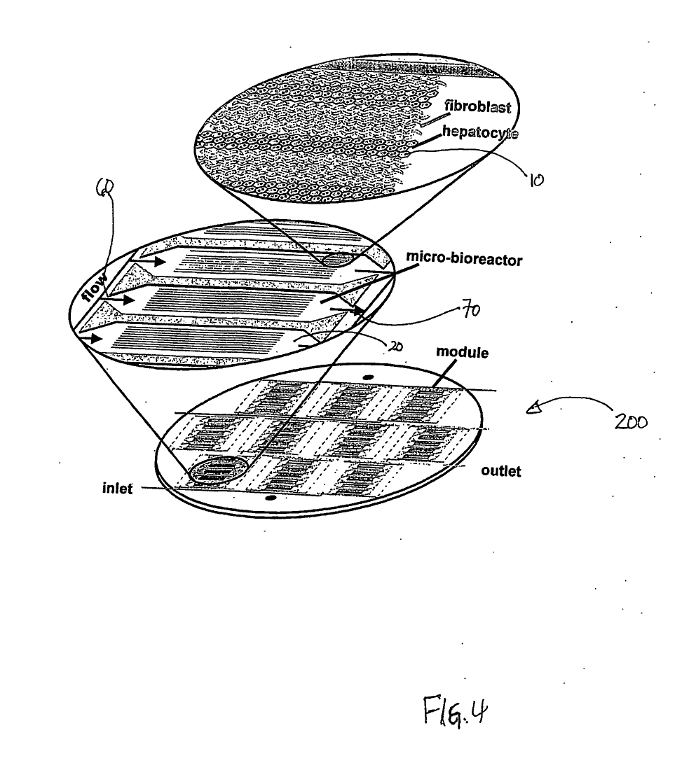

In a further embodiment, the bioreactor system 5 may be used in an array of bioreactor systems as depicted in FIG. 4. FIG. 4 is a schematic representation of a plurality of miniature bioreactor systems 5 in fluid communication. Depicted are inlet port 60 and outlet port 70 for each cell culture device 15. Cells 10 in each culture device 15 are grown on substrate 20 or a plurality of substrates 20.

Referring again to FIG. 3, one embodiment of a bioreactor 5 according to the disclosure has a tissue 10, which is seeded on top portion 30 of substrate 20. A cover chamber or housing 50 comprises at least one wall 55. The chamber/housing 50 comprises an inlet port 60 and outlet port 70. A tissue 10 can comprise (1) monolayer cell cultures (substantially homogenous for one cell type), mono-layer co-cultures of stromal and parenchymal cells, three-dimensional cultures comprising multi-functional cells, as well as all intermediate stages of cell/tissue growth and development during the culturing process.

The top portion 30 of substrate 20 sealingly engages chamber/housing 50 to create a flow space (depicted by the arrows in FIG. 3). The chamber/housing 50 comprises openings for fluid flow. Fluid supply tubes are provided at the inlet 60 and are in fluid communication with gas exchanger 100, pump 90, and fluid reservoir 80. Return tubes are provided at the outlet 70. Fluid circulation is maintained in the system using a pump 90 that can be any pump routinely used in cell culture systems including, for example, syringe pumps and peristaltic or other type of pump for delivery of fluid through the bioreactor.

Inlet port 60 and outlet ports 70 comprise fittings or adapters that mate tubing to maintain circulation of the fluid in the system. The fittings or adapters may be a Luer fitting, screw threads, or the like. The tubing fittings or adapters may be composed of any material suitable for delivery of fluid (including nutrient media) for cell culture. Such tubing fittings and adapters are known in the art. Typically, inlet port 60 and outlet port 70 comprise fittings or adapters that accept tubing having a desired inner diameter for the size of the reactor and the rate of fluid flow.

Substrate 20 can be made of any material suitable for culturing mammalian cells. For example, the substrate can be a material that can be easily sterilized such as plastic or other artificial polymer material, so long as the material is biocompatible. Substrate 20 can be any material that allows cells and/or tissue to adhere (or can be modified to allow cells and/or tissue to adhere) and that allows cells and/or tissue to grow in one or more layers. Any number of materials can be used to form the substrate 20, including, but not limited to, polyamides; polyesters; polystyrene; polypropylene; polyacrylates; polyvinyl compounds (e.g. polyvinylchloride); polycarbonate (PVC); polytetrafluoroethylene (PTFE); nitrocellulose; cotton; polyglycolic acid (PGA); cellulose; dextran; gelatin, glass, fluoropolymers, fluorinated ethylene propylene, polyvinylidene, polydimethylsiloxane, polystyrene, and silicon substrates (such as fused silica, polysilicon, or single silicon crystals), and the like. Also metals (gold, silver, titanium films) can be used.

Certain materials, such as nylon, polystyrene, and the like, are less effective as substrates for cellular and/or tissue attachment. When these materials are used as the substrate it is advisable to pre-treat the substrate prior to inoculation with cells in order to enhance the attachment of cells to the substrate. For example, prior to inoculation with stromal cells and/or parenchymal cells, nylon substrates should be treated with 0.1M acetic acid, and incubated in polylysine, FBS, and/or collagen to coat the nylon. Polystyrene could be similarly treated using sulfuric acid.

Where the in vitro generated artificial tissue is itself to be implanted in vivo, a biodegradable substrate such as polyglycolic acid, collagen, polylactic acid or hyaluronic acid should be used. Where the tissues are to be maintained for long periods of time or cryo-preserved, non-degradable materials such as nylon, dacron, polystyrene, polyacrylates, polyvinyls, teflons, cotton, and the like, may be used.

After a tissue has been grown in the bioreactor, it can be frozen and preserved in the bioreactor container itself. In one aspect, the tissue is preserved by reducing the temperature to about 4° C. Where the tissue is to be cryopreserved, cryopreservative is added through the fluid inlet ports, and then the inlet and outlet ports are sealed, providing a closed environment. The tissue can then be frozen in the bioreactor container, and thawed when needed. Methods for cryopreserving tissue will depend on the type of tissue to be preserved and are well known in the art.

The tissues and bioreactors of the disclosure can be used in a wide variety of applications. These include, but are not limited to, transplantation or implantation of the cultured artificial tissue in vivo; screening cytotoxic compounds, growth/regulatory factors, pharmaceutical compounds, and the like, in vitro; elucidating the mechanisms of certain diseases; studying the mechanisms by which drugs and/or growth factors operate; diagnosing and monitoring cancer in a patient; gene therapy and protein delivery; the production of biological products; and as the main physiological component of an extracorporeal organ assist device, to name a few. The tissues cultured by means of the bioreactors of the disclosure are particularly suited for the above applications, as the bioreactors allow the culturing of tissues having multifunctional cells. Thus, these tissues effectively simulate tissues grown in vivo.

In one embodiment, the bioreactors of the disclosure could be used in vitro to produce biological cell products in high yield. For example, a cell which naturally produces large quantities of a particular biological product (e.g. a growth factor, regulatory factor, peptide hormone, antibody, and the like) or a host cell genetically engineered to produce a foreign gene product, could be cultured using the bioreactors of the disclosure in vitro.

To use a bioreactor to produce biological products, a media flow having a concentration of solutes such as nutrients, growth factors and gases flows in through port 60 and out through port 70, over one surface of a tissue 10 seeded on substrate 20. The concentrations of solutes and nutrients (e.g., oxygen) provided are such that the tissue layer produces the desired biological product. Product is then excreted into the media flows, and can be collected from the effluent stream exiting through outlet port 70 using techniques that are well-known in the art.

As indicated above, reactors of different scales can be used for different applications. A large scale reactor can be used to study the effects of nutrient, drugs, and the like on tissue function (e.g., ischemia on the liver and its implications such as cellular hypoxic response an organ preservation). A high throughput reactor can be used for the evaluation of drugs for metabolism, toxicity and adverse xenobiotic interactions. It could also be used for the evaluation of potential cancer drugs and other pharmacological agents in variable oxygen environments. For example, miniaturized bioreactor system can be made into an array such as depicted in FIG. 4.

For growth of cells including, for example, hepatocytes and/or stromal cells, media containing solutes required for sustaining and enhancing tissue growth are pumped through inlet 60 to outlet port 70 in a fluid space defined by housing 50 and substrate 20. Solutes in the fluid media include nutrients such as proteins, carbohydrates, lipids, growth factors, as well as oxygen and other substances that contribute to cell and/or tissue growth and function. In particular, the oxygen gas concentration in the bioreactor system is regulated to maintain tissue morphology (e.g., zonation in liver tissue cultures). Such zonation promotes protein production by a tissue as described herein. The solutes in the media as well as those produced and release by cells in culture facilitate the development of multifunctional cells. As discussed above, the functional morphology and phenotypes of tissue parenchymal cells are governed by their exposure to the nutrients and oxygen present in the afferent fluid (e.g., nutrient) supply. In liver tissue in vivo, the liver receives substantial amounts of blood from the hepatic artery (rich in oxygen and poor in nutrients) and the hepatic portal vein (rich in nutrients coming from the gut organs and hormones such as insulin but poor in oxygen). The bioreactor system of the disclosure models this flow and nutrient/oxygen gradient from the inlet port to the outlet port.

The oxygen and nutrient gradients within the bioreactor drive parenchymal cell metabolism and contribute to the functional heterogeneity of the cells in the bioreactor. For example, as demonstrated in the specific examples below, by modulating oxygenation across liver tissue the tissue develops zonation regions characteristic of the zonations found in vivo. The bioreactor culture system of the disclosure allows for control of the microenvironment of cells in a cultured tissue by creating oxygen gradients that mimic in vitro the in vivo conditions.

The rate at which media is flowed through the bioreactor of the disclosure may depend on a variety of factors such as the size of the bioreactor, surface area of the tissue, type of tissue and particular application.

Isolated human hepatocytes are highly unstable in culture and are therefore of limited utility for studies on drug hepatotoxicity, drug-drug interaction, drug-related induction of detoxification enzymes, and other liver-based phenomena. The alternative approach is to employ animal experimentation to study the liver's response; however, there are many well-documented differences between animal and human metabolism that lead to inconclusive or inaccurate interpretation of animal data for human applications. The disclosure is an in vitro model of human liver tissue that can be utilized for pharmaceutical drug development, basic science research, and in the development of tissue for transplantation.

In one aspect, micropatterned cultures comprising parenchymal cells and stromal cells are used in the bioreactor system. In this aspect, the substrate is modified and prepared such that stromal cells are interspersed with the parenchymal cells. Using microfabrication techniques modified from the semiconductor industry, the substrate is modified to provide for spatially arranging parenchymal cells (e.g., (human hepatocytes) and supportive stromal cells (e.g., fibroblasts) in a miniaturizable format. Specifically, parenchymal cells (e.g., hepatocytes) can be prepared in circular islands of varying dimensions (36 μm, 100 μm, 490 μm, 4.8 mm, and 12.6 mm in diameter) surrounded by stromal cells (e.g., fibroblast such as murine 3T3 fibroblasts). Furthermore, parenchymal cell function may be modified by altering the pattern configuration. For example, hepatocyte detoxification functions are maximized at small patterns, synthetic ability at intermediate dimensions, while metabolic function and normal morphology were retained in all patterns.

As mentioned herein, in some instances the substrate may be modified to promote cellular adhesion and growth. For example, a glass substrate may be treated with protein (i.e., a peptide of at least two amino acids) such as collagen or fibronectin to assist cells in adhering to the substrate. In some embodiments, the proteinaceous material is used to define (i.e., produce) a micropattern. The micropattern produced by the protein serves as a “template” for formation of the cellular micropattern. Typically, a single protein will be adhered to the substrate, although two or more proteins may be used. Proteins that are suitable for use in modifying a substrate to facilitate cell adhesion include proteins to which specific cell types adhere under cell culture conditions. For example, hepatocytes are known to bind to collagen. Therefore, collagen is well suited to facilitate binding of hepatocytes. Other suitable proteins include fibronectin, gelatin, collagen type IV, laminin, entactin, and other basement proteins, including glycosaminoglycans such as heparin sulfate. Combinations of such proteins also can be used.

Using a combination of modified oxygen delivery and micropatterning of co-cultures can lead to a tissue model that can be optimized for specific physiologic functions including, for example, synthetic, metabolic, or detoxification function (depending on the function of interest) in hepatic cell cultures.

The use of the micropattern technology in combination with the bioreactor system of the disclosure allows for the development of microarray bioreactors as discussed above. Previous bioreactors were not amenable to miniaturization due in part to variable tissue organization due to reliance on self-assembly that underlie variations in nutrient and drug transport, and have uncharacterized stromal contaminants. Furthermore, previous random co-cultures have uncharacterized stromal cell population, have difficulty with microscopic imaging, have difficulty assessing cell number (due to proliferating cell populations) and display less liver-specific function than micropatterned co-cultures. The micropatterning of the cell types overcomes many of these difficulties.

In one aspect, the bioreactor utilizes co-cultures of cells in which at least two types of cells are configured in a micropattern on a substrate. By using micropatterning techniques to modulate the extent of heterotypic cell-cell contacts. In addition, co-cultures (both micropatterned co-cultures and non-micropatterned co-cultures) have improved stability and thereby allow chronic testing (e.g., chronic toxicity testing as required by the Food and Drug Administration for new compounds). Because micropatterned co-cultures are more stable than random cultures the use of co-cultures and more particularly micropatterned co-cultures provide a beneficial aspect to the cultures systems of the disclosure. Furthermore, because drug-drug interactions often occur over long periods of time the benefit of stable co-cultures allows for analysis of such interactions and toxicology measurements.

Typically, in practicing the methods of the disclosure, the cells are mammalian cells, although the cells may be from two different species (e.g., pigs, humans, rats, mice, and the like). The cells can be primary cells, or they may be derived from an established cell-line. Although any cell type that adheres to a substrate can be used in the methods and systems of the disclosure (e.g., parenchymal and/or stromal cells), exemplary combinations of cells for producing the co-culture include, without limitation: (a) human hepatocytes (e.g., primary hepatocytes) and fibroblasts (e.g., normal or transformed fibroblasts, such as NIH 3T3-J2 cells); (b) hepatocytes and at least one other cell type, particularly liver cells, such as Kupffer cells, Ito cells, endothelial cells, and biliary ductal cells; and (c) stem cells (e.g., liver progenitor cells, oval cells, hematopoietic stem cells, embryonic stem cells, and the like) and human hepatocytes and/or other liver cells and a stromal cell (e.g., a fibroblast). Other combination of hepatocytes, liver cells, and liver precursor cells.

In another aspect, certain cell types have intrinsic attachment capabilities, thus eliminating a need for the addition of serum or exogenous attachment factors. Some cell types will attach to electrically charged cell culture substrates and will adhere to the substrate via cell surface proteins and by secretion of extracellular matrix molecules. Fibroblasts are an example of one cell type that will attach to cell culture substrates under these conditions.

The methods and the bioreactors of the disclosure can be used for therapy and tissue testing. For example, a co-culture of hepatocytes and fibroblasts can be used as an implantable (in vivo) or extracorporeal (ex vivo) artificial liver for replacement of liver function (e.g., in response to diseases, infections, or trauma), or in in vitro assays of liver function (for example, for toxicology or basic research purposes). Similarly, such cultures can be used as a means to manufacture peptide compounds such as protein, enzymes, or hormones (e.g., albumin or clotting factors produced from hepatocytes).

Cells useful in the methods and to populate a bioreactor of the disclosure are available from a number of sources including commercial sources. For example, hepatocytes may be isolated by conventional methods (Berry and Friend, 1969, J. Cell Biol. 43:506-520) which can be adapted for human liver biopsy or autopsy material. Typically, a canula is introduced into the portal vein or a portal branch and the liver is perfused with calcium-free or magnesium-free buffer until the tissue appears pale. The organ is then perfused with a proteolytic enzyme such as a collagenase solution at an adequate flow rate. This should digest the connective tissue framework. The liver is then washed in buffer and the cells are dispersed. The cell suspension may be filtered through a 70 μm nylon mesh to remove debris. Hepatocytes may be selected from the cell suspension by two or three differential centrifugations.

For perfusion of individual lobes of excised human liver, HEPES buffer may be used. Perfusion of collagenase in HEPES buffer may be accomplished at the rate of about 30 ml/minute. A single cell suspension is obtained by further incubation with collagenase for 15-20 minutes at 37° C. (Guguen-Guillouzo and Guillouzo, eds, 1986, “Isolated and Culture Hepatocytes” Paris, INSERM, and London, John Libbey Eurotext, pp. 1-12; 1982, Cell Biol. Int. Rep. 6:625-628).

Hepatocytes may also be obtained by differentiating pluripotent stem cell or liver precursor cells (i.e., hepatocyte precursor cells). The isolated hepatocytes may then be used in the culture systems described herein.

Stromal cells include, for example, fibroblasts obtained from appropriate sources as described further herein. Alternatively, the stromal cells may be obtained from commercial sources or derived from pluripotent stem cells using methods known in the art.

Fibroblasts may be readily isolated by disaggregating an appropriate organ or tissue which is to serve as the source of the fibroblasts. This may be readily accomplished using techniques known to those skilled in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase, dispase and the like. Mechanical disruption can also be accomplished by a number of methods including, but not limited to, the use of grinders, blenders, sieves, homogenizers, pressure cells, or insonators. For a review of tissue disaggregation techniques, see Freshney, Culture of Animal Cells. A Manual of Basic Technique, 2d Ed., A. R. Liss, Inc., New York, 1987, Ch. 9, pp. 107-126.

Once the tissue has been reduced to a suspension of individual cells, the suspension can be fractionated into subpopulations from which the fibroblasts and/or other stromal cells and/or elements can be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis, fluorescence-activated cell sorting, and the like. For a review of clonal selection and cell separation techniques, see Freshney, Culture of Animal Cells. A Manual of Basic Techniques, 2d Ed., A. R. Liss, Inc., New York, 1987, Ch. 11 and 12, pp. 137-168.

The isolation of fibroblasts can, for example, be carried out as follows: fresh tissue samples are thoroughly washed and minced in Hanks balanced salt solution (HBSS) in order to remove serum. The minced tissue is incubated from 1-12 hours in a freshly prepared solution of a dissociating enzyme such as trypsin. After such incubation, the associated cells are suspended, pelleted by centrifugation and plated onto culture dishes. All fibroblasts will attach before other cells, therefore, appropriate stromal cells can be selectively isolated and grown. The isolated fibroblasts can then be used in the culture systems of the disclosure.

For example, and not by way of limitation, endothelial cells may be isolated from small blood vessels of the brain according to the method of Larson et al. (1987, Microvasc. Res. 34:184) and their numbers expanded by culturing in vitro using the bioreactor system of the disclosure. Silver staining may be used to ascertain the presence of tight junctional complexes specific to small vessel endothelium and associated with the “barrier” function of the endothelium.

Suspensions of pancreatic acinar cells may be prepared by an adaptation of techniques described by others (Ruoff and Hay, 1979, Cell Tissue Res. 204:243-252; and Hay, 1979, in, “Methodological Surveys in Biochemistry Vol. 8, Cell Populations.” London, Ellis Hornwood, Ltd., pp. 143-160). Briefly, the tissue is minced and washed in calcium-free, magnesium-free buffer. The minced tissue fragments are incubated in a solution of trypsin and collagenase. Dissociated cells may be filtered using a 20 μm nylon mesh, resuspended in a suitable buffer such as Hanks balanced salt solution (HBSS), and pelleted by centrifugation. The resulting pellet of cells can be resuspended in minimal amounts of appropriate media and inoculated onto a substrate for culturing in the bioreactor system of the disclosure. The pancreatic cells may be cultured with stromal cells such as fibroblasts. Acinar cells can be identified on the basis of zymogen droplet inclusions.

Cancer tissue may also be cultured using the methods and bioreactor culture system of the disclosure. For example, adenocarcinoma cells can be obtained by separating the adenocarcinoma cells from stromal cells by mincing tumor cells in HBSS, incubating the cells in 0.27% trypsin for 24 hours at 37° C. and further incubating suspended cells in DMEM complete medium on a plastic petri dish for 12 hours at 37° C. Stromal cells selectively adhered to the plastic dishes.

The tissue cultures and bioreactors of the disclosure may be used to study cell and tissue morphology. For example, enzymatic and/or metabolic activity may be monitored in the culture system remotely by fluorescence or spectroscopic measurements on a conventional microscope. In one aspect, a fluorescent metabolite in the fluid/media is used such that cells will fluoresce under appropriate conditions (e.g., upon production of certain enzymes that act upon the metabolite, and the like). Alternatively, recombinant cells can be used in the cultures system, whereby such cells have been genetically modified to include a promoter or polypeptide that produces a therapeutic or diagnostic product under appropriate conditions (e.g., upon zonation or under a particular oxygen concentration). For example, a hepatocyte may be engineered to comprise a GFP (green fluorescent protein) reporter on a P450 gene (CYPIA1). Thus, if a drug activates the promoter, the recombinant cell fluoresces. This is useful for predicting drug-drug interactions that occur due to upregulation in P450s.

The tissue cultures and bioreactors of the disclosure may be used to in vitro to screen a wide variety of compounds, such as cytotoxic compounds, growth/regulatory factors, pharmaceutical agents, and the like, to identify agents that modify cell (e.g., hepatocyte) function and/or cause cytotoxicity and death or modify proliferative activity or cell function. For example, the culture system may be used to test adsorption, distribution, metabolism, excretion, and toxicology (ADMET) of various agents. To this end, the cultures are maintained in vitro under a desired oxygen concentration and exposed to a compound to be tested. The activity of a compound can be measured by its ability to damage or kill cells in culture or by its ability to modify the function of the cells (e.g., in hepatocytes the expression of P450, and the like). This may readily be assessed by vital staining techniques, ELISA assays, immunohistochemistry, and the like. The effect of growth/regulatory factors on the cells (e.g., hepatocytes, endothelial cells, epithelial cells, pancreatic cells, astrocytes, muscle cells, cancer cells) may be assessed by analyzing the cellular content of the culture, e.g., by total cell counts, and differential cell counts or by metabolic markers such as MTT and XTT. This may also be accomplished using standard cytological and/or histological techniques including the use of immunocytochemical techniques employing antibodies that define type-specific cellular antigens. The effect of various drugs on normal cells cultured in the culture system may be assessed. For example, drugs that affect cholesterol metabolism, e.g., by lowering cholesterol production, could be tested on a liver culture system.

On advantage of the bioreactor and culture systems of the disclosures (e.g., a single as well as an array of bioreactors of the invention) is that the cells in such a bioreactor or culture system are substantially homogenous and autologous so you can do many experiments on the same biological background. Furthermore, in vivo testing suffers from animal-to-animal variability and is limited by the number of conditions or agents that can be tested on a given subject.

The cytotoxicity to cells in culture (e.g., human hepatocytes) of pharmaceuticals, anti-neoplastic agents, carcinogens, food additives, and other substances may be tested by utilizing the bioreactor culture system of the disclosure.

First, a stable, growing culture is established within the bioreactor system having a desired oxygen utilization and gradient such that ideal zonation is established. Then, the cells/tissue in the culture are exposed to varying concentrations of a test agent. After incubation with a test agent, the culture is examined by phase microscopy to determine the highest tolerated dose—the concentration of test agent at which the earliest morphological abnormalities appear. Cytotoxicity testing can be performed using a variety of supravital dyes to assess cell viability in the liver culture system, using techniques well-known to those skilled in the art.

Once a testing range is established, varying concentrations of the test agent can be examined for their effect on viability, growth, and/or morphology of the different cell types by means well known to those skilled in the art.

Similarly, the beneficial effects of drugs or biologics may be assessed using the bioreactor culture system. For example, growth factors, hormones, or drugs which are suspected of having the ability to enhance cell or tissue function, formation or activity can be tested. In this case, stable cultures are exposed to a test agent. After incubation, the cultures are examined for viability, growth, morphology, cell typing, and the like, as an indication of the efficacy of the test substance. Varying concentrations of the drug may be tested to derive a dose-response curve.

The culture systems of the disclosure may be used as model systems for the study of physiologic or pathologic conditions. For example, in a specific embodiment, a liver culture system can be optimized to act in a specific functional manner as described herein by modifying the oxygen delivery and gradient in the bioreactor system.

The bioreactor culture system may also be used to aid in the diagnosis and treatment of malignancies and diseases. For example, a biopsy of a tissue (such as, for example, a liver biopsy) may be taken from a subject suspected of having a malignancy or other disease or disorder. The biopsy cells can then be cultured in the bioreactor system under appropriate oxygen concentrations where the activity of the cultured cells can be assessed using techniques known in the art. In addition, such biopsy cultures can be used to screen agent that modify the activity in order to identify a therapeutic regimen to treat the subject. For example, the subject's tissue culture could be used in vitro to screen cytotoxic and/or pharmaceutical compounds in order to identify those that are most efficacious; i.e. those that kill the malignant or diseased cells, yet spare the normal cells. These agents could then be used to therapeutically treat the subject.

Similarly, the beneficial effects of drugs may be assessed using the culture system in vitro; for example, growth factors, hormones, drugs which enhance hepatocyte formation or activity can be tested. In this case, stable micropattern cultures may be exposed to a test agent. After incubation, the micropattern cultures may be examined for viability, growth, morphology, cell typing, and the like as an indication of the efficacy of the test substance. Varying concentrations of the drug may be tested to derive a dose-response curve.

The culture systems of the invention may be used as model systems for the study of physiologic or pathologic conditions. For example, in a specific embodiment, the culture system can be optimized to act in a specific functional manner as described herein by modifying the oxygen concentration at the inlet and outlet to provide a gradient across the tissue. In another aspect, the oxygen gradient is modified along with the density and or size of a micropattern of cells in the culture system.

The various techniques, methods, and aspects of the invention described above can be implemented in part or in whole using computer-based systems and methods. Particularly, the regulation of desired pO₂ values with in a fluid media can be regulated by a computer system based upon the information obtained from O₂ sensors within the bioreactor system 5. Additionally, computer-based systems and methods can be used to augment or enhance the functionality described above, increase the speed at which the functions can be performed, and provide additional features and aspects as a part of or in addition to those described elsewhere in this document. Various computer-based systems, methods and implementations in accordance with the above-described technology are presented below.

A processor-based system can include a main memory, preferably random access memory (RAM), and can also include a secondary memory. The secondary memory can include, for example, a hard disk drive and/or a removable storage drive, representing a floppy disk drive, a magnetic tape drive, an optical disk drive, etc. The removable storage drive reads from and/or writes to a removable storage medium. Removable storage medium refers to a floppy disk, magnetic tape, optical disk, and the like, which is read by and written to by a removable storage drive. As will be appreciated, the removable storage medium can comprise computer software and/or data.

In alternative embodiments, the secondary memory may include other similar means for allowing computer programs or other instructions to be loaded into a computer system. Such means can include, for example, a removable storage unit and an interface. Examples of such can include a program cartridge and cartridge interface (such as the found in video game devices), a movable memory chip (such as an EPROM or PROM) and associated socket, and other removable storage units and interfaces, which allow software and data to be transferred from the removable storage unit to the computer system.

The computer system can also include a communications interface. Communications interfaces allow software and data to be transferred between computer system and external devices. Examples of communications interfaces can include a modem, a network interface (such as, for example, an Ethernet card), a communications port, a PCMCIA slot and card, and the like. Software and data transferred via a communications interface are in the form of signals, which can be electronic, electromagnetic, optical or other signals capable of being received by a communications interface (e.g., information from O₂ sensors). These signals are provided to communications interface via a channel capable of carrying signals and can be implemented using a wireless medium, wire or cable, fiber optics or other communications medium. Some examples of a channel can include a phone line, a cellular phone link, an RF link, a network interface, and other communications channels.

In this document, the terms “computer program medium” and “computer usable medium” are used to refer generally to media such as a removable storage device, a disk capable of installation in a disk drive, and signals on a channel. These computer program products are means for providing software or program instructions to a computer system. In particular, the disclosure includes instructions on a computer readable medium for calculating the proper O₂ concentrations to be delivered to a bioreactor system comprising particular dimensions and cell types.

Computer programs (also called computer control logic) are stored in main memory and/or secondary memory. Computer programs can also be received via a communications interface. Such computer programs, when executed, enable the computer system to perform the features of the disclosure including the regulation of desired pO₂ values within a bioreactor system.

In an embodiment where the elements are implemented using software, the software may be stored in, or transmitted via, a computer program product and loaded into a computer system using a removable storage drive, hard drive or communications interface. The control logic (software), when executed by the processor, causes the processor to perform the functions of the invention as described herein.

In another embodiment, the elements are implemented primarily in hardware using, for example, hardware components such as PALs, application specific integrated circuits (ASICs) or other hardware components. Implementation of a hardware state machine so as to perform the functions described herein will be apparent to person skilled in the relevant art(s). In yet another embodiment, elements are implanted using a combination of both hardware and software.

The working examples provided below are to illustrate, not limit, the disclosure. Various parameters of the scientific methods employed in these examples are described in detail below and provide guidance for practicing the disclosure in general.

In these particular working examples, hepatocytes are co-cultured with fibroblasts. Similar methods can be used to co-culture other combinations of cells. These experiments demonstrate that one or more cell types can be cultured in a bioreactor system with a controlled oxygen to obtain cells that are phenotypically similar to corresponding cells in vivo as well as tissue that is morphologically similar to tissue in vivo. Although the invention has been generally described above, further aspects of the invention will be apparent from the specific disclosure that follows, which is exemplary and not limiting.

### EXAMPLES

Hepatocyte Isolation And Culture. Primary rat hepatocytes were isolated and purified from 2- to 3-month-old adult female Lewis rates (Charles River Laboratories, Williminton, Mass.) weighing 180-200 g, by a modified procedure of Seglen (1976). Prior to being seeded, microscope slides (38 mm×75 mm) were washed in ethanol, rinsed thoroughly with sterile water, and incubated for 1 h at 37° C. in a type I collagen solution (0.11 mg/mL). Hepatocytes were cultured on slides to confluency with duplicate seedings of 1.5 to 3×10⁶ cells and gentle shaking every 15 minutes for 1 h after each seeding in media consisting of Dulbeccos's Modified Eagle Medium (DMEM, GibcoBRL, Rockville, Md.) with 10% fetal bovine serum, supplemented with insulin, hydrocortisone, and antibiotics. Two hours after seeding media was changed to a serum-free formulation of DMEM/Hams' F12 with insulin (5 μg/mL), dexamethasone (10⁻⁸ M), linoleic acid (5 μg/ml), trace elements (ZnSO₄, 10⁻¹⁰ M, CuSO₄, 10⁻⁷ M, H₂SeO₃, 3×10⁻¹⁰ M), and antibiotics. Culture media was buffered with bicarbonate under 5% CO₂ before use in the flow chamber and with 20 mM HEPES during chamber experiments. All experiments were performed on day 1 or 2 post-isolation.

Co-cultures. Hepatocytes were isolated as above. Following collagen adsorption, 1.5×10⁶ hepatocytes were seeded on microscope slides and allowed to attach for 2 hours, at which point media was replaced. Cocultures were created by adding 750,000 J2-3T3 fibroblasts/slide 24 hours after hepatocyte seeding. Cultures were allowed to stabilize to day 5 with media changes every 48 hours and experiments were carried out between days 5-7 post-isolation. Bioreactor cultures were perfused with media supplemented with various chemicals to evaluate regional changes in protein expression and toxicity. Induction of CYP2B and CYP3A was carried out by adding 200 μM phenobarbital or 5 μM, respectively. Additionally, EGF was added at a concentration of 2 nM to examine its role in modulating CYP expression. Toxicity experiments for both static and perfused cultures were performed by adding APAP ranging from 5-40 mM to culture media for 24 hours. Images were obtained using a Nikon Eclipse TE300 inverted microscope, CCD camera (CoolSnap HQ, Roper Scientific), and Metamorph Image Analysis System (Universal Imaging). Metabolic activity was evaluated using MTT-stained cultures by obtaining full-field images using a Nikon Coolpix 3100 digital camera and also a series low-magnification images from the Nikon TE200. Relative viability was determined from the optical density of triplicate images at 5 positions along the length of the slide.

Bioreactor And Flow Circuit. A flat-plate bioreactor was designed to conduct experiments using 38×75 mm microscope slides. A polycarbonate block was milled to create rectangular inlet and outlet ports in a 100 μm (±10 μm) recess over which a chamber slide could be placed. Slides were sealed in the chamber with inert silicone lubricant (Dow Corning, Midland, Mich.) and a stainless steel bracket with six screws. The flow field dimensions used in model calculation were 28 mm (width)×55 mm (length)×100 μm (height). After assembly, the chamber was inserted to the flow circuit containing a media reservoir, gas exchange, O₂ probe, and syringe pump. Pressure-driven flow was continuous using a programmable push-pull syringe pump (Harvard Apparatus, Holliston, Mass.). Media was equilibrated with 10% or 21% O₂ in a gas exchanger made with gas-permeable silastic tubing. A miniature Clark-type electrode was placed at the chamber outlet to measure dissolved O₂ concentration (Microelectrodes, Inc., Bedford, N.H.). Electrode zeroing was carried out periodically while calibration at the inlet pO₂ was carried out prior to each experiment. Experimental flow rates of recirculating media varied from 0.2 to 4 mL/min. All flow circuit components except for the syringe pump were housed in a PID-controlled incubator maintained at 37° C.

Microscope And Immunohistochemistry. Images were obtained using a Nikon Eclipse TE200 inverted microscope, SPOT digital camera (Diagnostic Instruments, Sterling Heights, Mich.), and Metamorph Image Analysis System (Universal Imaging, Downingtown, Pa.). Viability was assessed by fluorescence after 24 h of perfusion using Hoechst dye 33258 (nuclear: ex365/em458), fluoresce in diacetate (viable: ex494/em516), and propidium iodide (non-viable: ex536/em617). Percent viability was determined by calculating total non-viable cell number and total cell number averaged from 3 fields of the chamber inlet, midline, and outlet.

Regional hypoxia in hepatocyte cultures was shown using the Hypoxyprobe Kit (NPI, Inc. Belmont, Mass.). This kit uses a probe, pimonidazole hydrochloride, which forms adducts with cellular proteins when pO₂ is below 10 mmHg. Cultures were perfused with media supplemented with Hypoxyprobe-1 (0.11 mM) for 3 h and then fixed with 4% paraformaldehyde in PBS. Samples were incubated with monoclonal antibody specific to Hypoxyprobe-1. Secondary staining methods were carried out using a DAKO labeled Streptavidin-Biotin staining kit (DAKO Corporation, Carpinteria, Calif.).

Zonal Induction. Historically, recapitulation of periportal-like and perivenous-like cell populations in vitro requires simultaneous stimulus by soluble factors and oxygen. Bioreactor cultures were perfused with media and allowed to reach steady state before the addition of inductive agents of glucagon for PEPCK up-regulation or phenobarbitol (PB) and EGF for CYP2B1 induction. Media supplemented with 10 nM glucagon was perfused for 8 h allowing for the cAMP-dependent induction of PEPECK before cell lysis. Media with 0.75 mM PB and 0.16 nM EGF was perfused for 36 h to induced CYB2B expression before cell lysis. Cell lysates were collected at the end of each experiment for electrophoretic analysis.

Western Blot Analysis. Primary rat hepatocytes from 6-well plates or chamber slides were lysed and scraped in SDS buffered (10 mM Tris/HCl (ph 7.4), 0.1% SDS). Samples were added to microcentrifuge tubes with 5 μl of PMSF, homogenized with a pestle, and centrifuged at 16,200 g for 5 minutes. Total protein content in the supernatant was determined using the DC protein assay (Bio-Rad, Hercules, Calif.) and used to normalize sample loading. Samples prepared in sample buffer were loaded (10-20 μg/well) for electrophoresis on a 10% polyacrylamide gel. After overnight transfer on to a PVDF membrane, blots were incubated with a blocking buffered (20 mM Tris-Cl (pH 7.4), 500 mM NaCl, 0.1% Tween 20, and 5% (w/v) milk powder) and washed with buffer without milk powder, incubation for 1 h with primary antibody against rat PEPCK or rat CYP2B (Genset, Woburn, Mass.) was followed by washing and incubation with either anti-sheep (PEPCK) or anti-goat (CYP2B) HRP-conjugated secondary antibody for 45 minutes. After washing, the Pierce SuperSignal chemiluminescence reagent was used for detection. All electrophoresis gels were run with molecular mass markers to verify PEPCK bands (67 kDa) or CYP2B1/2 bands (56 kDa). Optical density measurements were obtained using scanned blot images with 1d gelscan software (Metamorph).

Statistics And Data Analysis. Statistical analysis and model computations were performed using MathCAD (Mathsoft, Inc., Cambridge, Mass.), which provides a symbolic interface for evaluating the analytical solution (Eq. (7)). Mathlab (Mathworks, Inc., Natick, Mass.) was used to formulate and analyze the numerical solution. Model and experimental data were plotted using Sigmaplot (SPSS, Inc., San Rafael, Calif.). Error was reported at the standard deviation of the mean and statistical significance was determined using one-way (ANOVA (P<0.05).

Bioreactor Model. The transport of oxygen in a parallel-plat bioreactor can be modeled using the equation of continuity for a binary system. By assuming steady-state transport in a uniform flow field in the x-direction and lateral diffusion in the y direction, the non-dimensional governing equation is obtained (Eq. 1):

$$\frac{\partial \hat{c}}{\partial \hat{x}} = \frac{α}{Pe} \frac{{\partial }^{2} \hat{c}}{\partial {\hat{y}}^{2}} , 0 \leq \hat{x} \leq 1 , 0 \leq \hat{y} \leq 1 , \qquad ( 1 )$$

where ĉ is the dimensionless concentration with respect inlet O₂ concentration, cᵢₙ(ĉ=[c−cᵢₙ]/cᵢₙ), and \hat{x} and ŷ are non-dimensionalized using the chamber height (H) and chamber length (L) according to \hat{x}=x/L and ŷ/H. The Peclet number, a ratio of convective and diffusive transport, is defined as Pe−uₘH/D, where uₘ is the mean velocity, D is oxygen diffusivity, and α=L/H. The boundary conditions are given in Eqs. (2-4):

$$\begin{matrix}\frac{\partial \hat{c}}{\partial \hat{y}} ( \hat{x} , 0 ) = 0 , 0 \leq \hat{x} \leq 1 , & ( 2 ) \\ \frac{\partial \hat{c}}{\partial \hat{y}} ( \hat{x} , 1 ) = - Da , 0 \leq \hat{x} \leq 1 , & ( 3 ) \\ \hat{c} ( 0 , \hat{y} ) = 0 , 0 \leq \hat{y} \leq 1 , & ( 4 )\end{matrix}$$

Inherently, boundary conditions assume no oxygen flux at the top of the chamber, constant flux at the cell surface, and a constant inlet oxygen concentration. The Damkohler number (Da), the dimensionless oxygen flux, is the ratio of the oxygen uptake rate and diffusion rate as shown in Eq. (5):

$$Da = \frac{{pV}_{max} H}{{Dc}_{in}} , \qquad ( 5 )$$

where p is the cell density and Vₘₐₓ is the maximal oxygen uptake rate. The model parameters used in calculation are listed in Table 1.

TABLE 1
Modeling Parameters (Foy et al., 1994)

| Parameter | Value | Units |
|---|---|---|
| D, O₂ diffusivity | 2 × 10⁻⁵ | cm²/s |
| Vₘₐₓ, max. O₂ uptake | 0.38 | nmol/s/10⁶ cells |
| Kₘ, Michaelis constant | 5.6 | mmHg |
| P, cell density | 1.7 × 10⁵ | Cells/cm² |
| Cᵢₙ, inlet O₂ conc. | 90-190 | nmol/mL |
| Q, volumetric flow | 0.3-3 | mL/min |
| rate | | |
| H, height | 100 | μm |
| W, width | 2.8 | cm |
| L, length | 5.5 | cm |

Eqs. (1-4) constitute a linear, homogenous differential equation with a non-homogeneous boundary condition that may be solved analytically. It is assumed that the solution is a combination of the convection-free solution and a homogenous convection-diffusion solution as given by:
ĉ(\hat{x},ŷ)=û(\hat{x},ŷ(+\hat{v}(\hat{x},ŷ). (6)
The convection-free solution, û(\hat{x},ŷ), is a polynomial expression that satisfies Eq. (1) and the boundary conditions. The second term, \hat{v}(\hat{x},ŷ), is derived by applying Fourier's method. The complete solution for the oxygen concentration profile is shown in Eq. (7).

$$\hat{c} ( \hat{x} , \hat{y} ) = Da [ \frac{1 - 3 {\hat{y}}^{2}}{6} - \frac{α}{Pe} \hat{x} + \frac{2}{{π}^{2}} {\sum }_{n = 1}^{∞} \frac{{( - 1 )}^{n}}{ {n}^{2}} exp ( - \frac{α {n}^{2} {π}^{2} \hat{x}}{Pe} ) cos ( n {π}^{2} ) ] . \qquad ( 7 )$$

The boundary condition given in Eq. (3) assumes constant oxygen uptake along the entire length of the chamber. Michaelis-Menten kinetics more accurately predicts oxygen uptake in rat hepatocytes, especially at low oxygen concentrations. Thus, a numerical solution for Eq. (1) was obtained using a Crank-Nicholson finite defining scheme, with first-order discretization at the boundaries and an explicit approximation of the Michaelis-Menten equation. Numerical results were compared to the closed form solution (Eq. (7)) to ensure consistency.

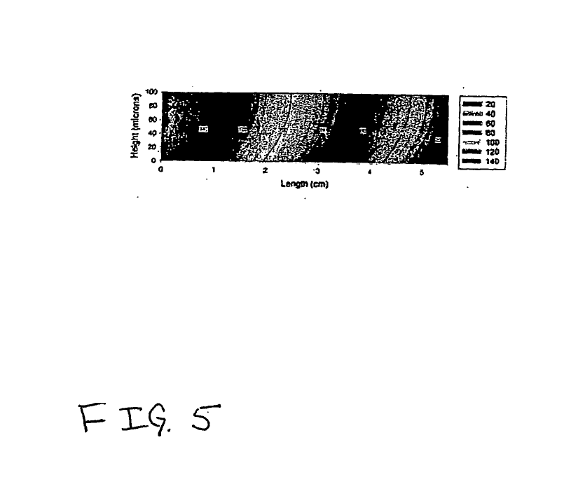

Model Output. The objective of the perfusion experiments was to impose a controlled oxygen gradient over the culture hepatocytes in order to modulate their function. Eq. (7) was used to predict the oxygen concentration profile along the length of the chamber. FIG. 5 demonstrates a contour plot of the O₂ distribution in the cross-section of the bioreactor (inlet pO₂=158 mmHg, Q=0.35 mL/min). The oxygen profile can be seen as a combination of oxygen diffusion to the cell surface with constant uptake and convection in the x direction. As the Peclet number is dependent on flow rate (proportional to mean longitudinal velocity, vₘ), it follows from the governing equation (Eq. (1)) that convective transport would dominate with increasing flow rate while diffusion transport becomes more important as flow approaches zero.

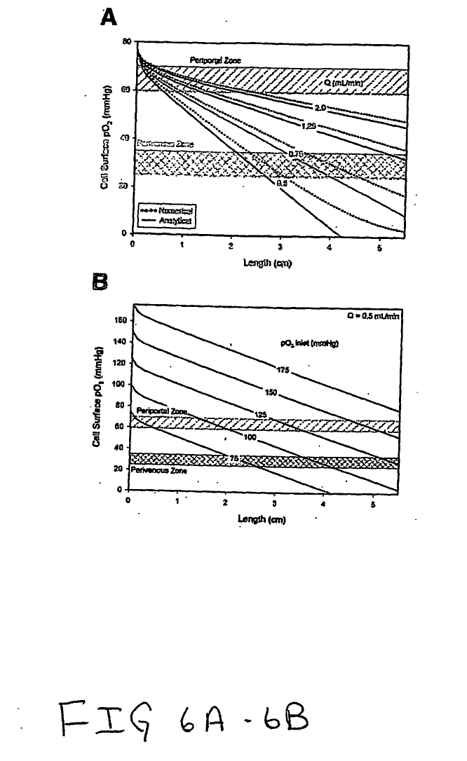

As a result, it is expected that lower flow rates allows sufficient diffusion to surface to induce oxygen gradients along the length of the bioreactor. FIG. 6A shows the flow rate dependence of oxygen concentration at the cell surface. Two regions are depicted that correspond to typical physiologic oxygen partial pressures found in the periportal zone (60-70 mmHg) and perivenous zone (25-35 mmHg) of the liver. Under optimal operating conditions, the transition from a periportal to a perivenous oxygen environment would occur at the midline (2.25 cm) and cell surface oxygen concentration would not drop below a crucial value of 5 mmHg. Given an inlet oxygen concentration of 76 mmHg (10% O₂), the optimal range of volumetric flow rate is 0.5 to 0.75 mL/min where the final 50% of chamber length could be exposed to perivenous oxygen levels (<35 mmHg). Similar analysis for an oxygen inlet pO₂ of 158 mmHg (21% O₂) indicates that operating at 0.3 mL/min subjects 25% of the chamber near the outlet to perivenous levels without hypoxia.

FIG. 6A also emphasizes the differences between the analytical and numerical solutions of Eq. (1). Deviations occur most significantly in regions of low pO₂ due to the assumption in the analytical solution that oxygen consumption is independent of concentration (Eq. (4)). With the application of Michaelis-Menten oxygen uptake kinetics at the boundary, the numerical solution accounts for concentration-dependent changes in oxygen demand. When the bioreactor is operated without oxygen limitations, such as is the case at 2.0 mL/min and above, the constant oxygen uptake assumption holds and better correlation is seen between analytical and numerical solutions.

Inlet oxygen concentration is another system parameter that may be used to modify bioreactor conditions. FIG. 6B shows the dependence of cell-surface oxygen concentrations on inlet concentration at a fixed flow rate of 0.5 mL/min. As shown, the slope of the oxygen gradients not affected, but changing inlet concentration shifts the absolute magnitude linearly. Though increasing the inlet oxygen concentration offers a wider range of operating conditions, physiological levels of oxygen below 90 mmHg are effectively applied across the entire culture with lower inlet concentrations. Experiments presented herein were performed with inlet partial pressures ranging from 76 to 158 mmHg.

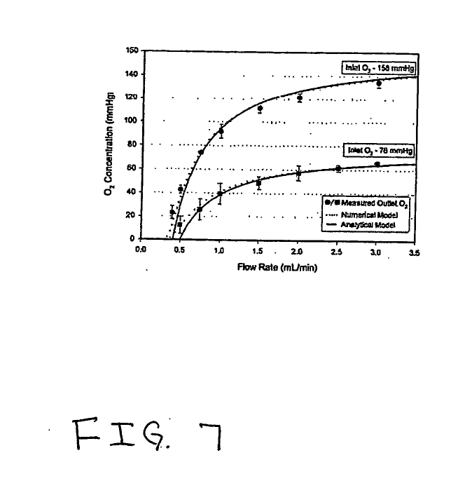

To verify the presence of oxygen gradients in the hepatocyte bioreactor, outlet oxygen levels were monitored and compared to predicted values. Outlet oxygen tension was measured as a function of flow rate, ranging from 0.4 to 3 mL/min. In single experiments, flow rates were changed every 15-30 minutes and allowed to reach steady state, at which point O₂ levels were recorded. By way of observation, output pO₂ levels became steady 2-3 minutes after a change in flow rate. In addition, experiments were conducted over a 4 hour period, at eth conclusion of which electrode drift was assessed and found to be less than 5%. Measured values were plotted against model predictions for two separate inlet oxygen conditions: 76 and 158 mmHg (FIG. 7). Results are the average and standard deviation of three separate validation experiments. Measured oxygen concentration correlated well with the analytical and numerical models. At lower flow rates and lower oxygen partial pressures, the numerical solution was a better estimation of outlet pO₂, as expected. Increased error in measurements was also noted at lower flow rates and may be due to electrode limitation.

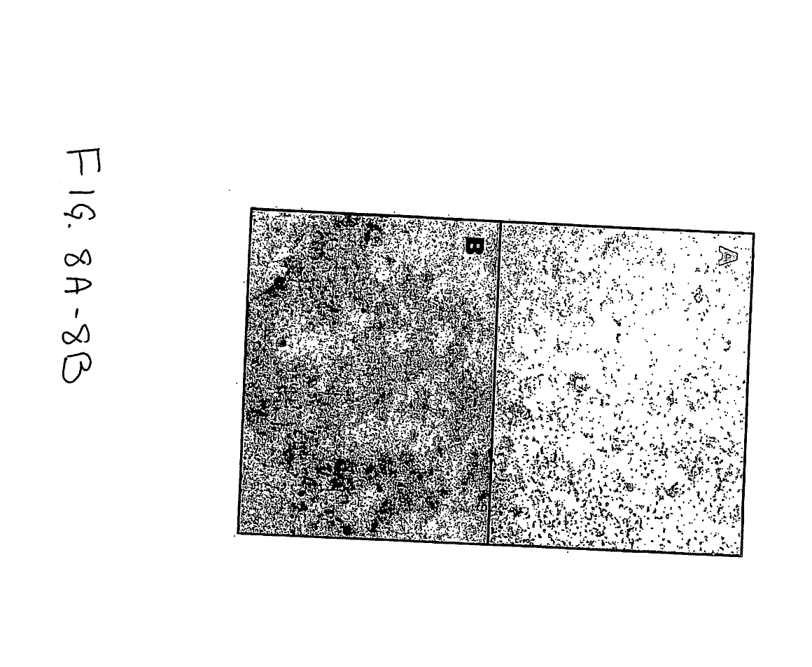

Bioreactor cultures were subjected to a gradient that predicted a hypoxic environment (pO₂<10 mmHg) to 50% of the bioreactor culture. Procedures were followed for application of the Hypoxyprobe kit with an inlet pO₂ of 76 mmHg and flow rate of 0.3 mL/min. In general, staining intensity indicating hypoxia gradually increased along eh length of the chamber. Bright-field images in FIG. 8 showed a significant increase hypoxia in the outlet t region (B) over the inlet (A).

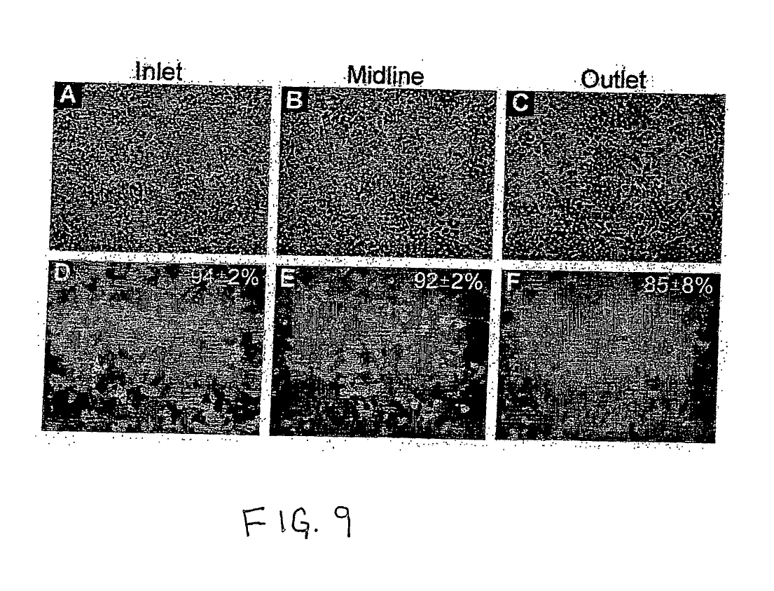

To evaluate possible hepatocyte necrosis due to decreased oxygen availability, bioreactor cultures were perfused for 24 h at 0.35 mL/min with 158 mmHg inlet pO₂. The predicted outlet pO₂ under these conditions is 8 mmHg, and measured levels were 15±3 mmHg after 4 h. After 24 h, images were acquired to assess morphology and viability. Phase images at the inlet, midline, and outlet showed that normal polygonal morphology and bile canaliculi were maintained (FIG. 9A, C, E). Fields from each of the three regions were taken to quantitate viability. The average and standard deviation from three fields are shown in FIGS. 9B, D, and F. Results indicated that over a 24-h period viability at the outlet was 85% but statistically was not significantly different from the inlet and middle regions. It was anticipated that these moderate changes in viability would not have an effect on cellular response to zonal induction of PEPCK and CYP2B.

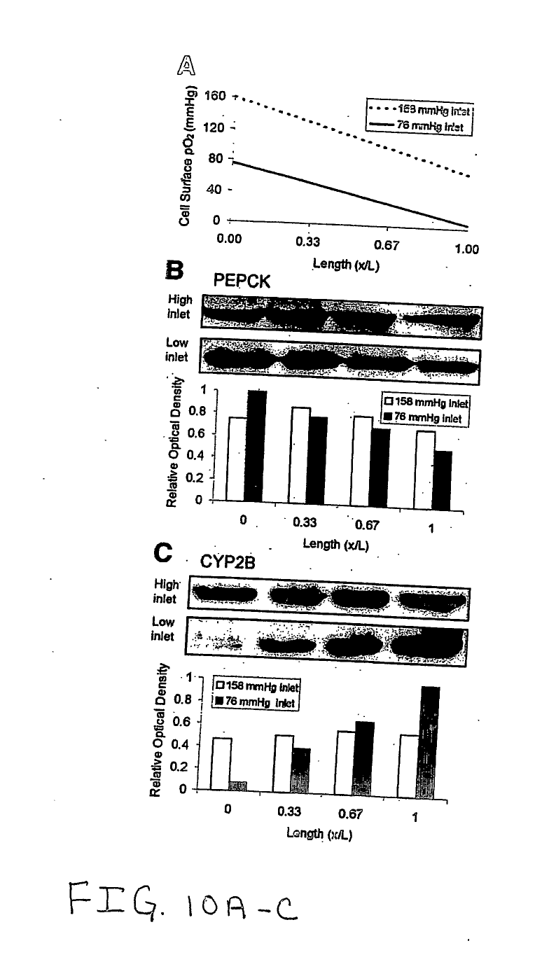

In vivo, PEPCK is predominately found in periportal regions that contain higher O₂, levels. In the bioreactor system, higher PEPCK levels would be expected in the inlet region when operating with a media flow rate of 0.5 mL/min, which results in a cell surface oxygen gradient of 76 to 5 mmHg from inlet to outlet (FIG. 10A). Western blot analysis of 4 separate bioreactor regions showed maximal PEPCK protein levels at the inlet decreasing to half maximal at the outlet (FIG. 10B). The depletion of O₂ in the bioreactor was responsible for the oxygen gradient, but the depletion of glucagon, which up-regulates PEPCK expression, may also have contributed to the regional variations in PEPCK. To evaluate the possibility of a glucagon gradient contributing to a PEPCK gradient, the bioreactor was operated with inlet pO₂ of 158 mmHg and the same flow rate, 0.5 ml/min. Under this supraphysiologic gradient, the heterogenous induction of PEPCK was abrogated, indicating that oxygen was likely to be the primary modulator of differential PEPCK protein levels in this system. In addition, experiments conducted with higher flow rates resulting in nominal oxygen gradients also showed relatively uniform PEPCK induction.

Similarly, low oxygen environments are thought to contribute to pericentral localization of CYP2B in vivo. The heterogeneous induction of CYP2B in the bioreactor was carried under a physiologic oxygen gradient with 76 mmHg inlet pO₂ and 0.5 mL/min (FIG. 10A). CYP2B levels were minimal at the chamber inlet and steadily increase to maximal induction in the low O₂ outlet region (FIG. 10C). EGF has been shown to be an inhibitor of CYP2B induction, and as is the case with glucagon, EGF depletion may result in a decreasing EGF gradient. Hence, the observed results may result, in part, from minimized inhibitory effects of CYP2B expression in the outlet region. To test this possibility, supraphysiologic gradients were imposed under the same flow rate that produced graded CYP2B levels and where EGF gradients were presumably similar. Under these conditions and in additional experiments at high flow rate without significant oxygen gradients, uniform CYP2B levels were observed. Therefore, physiologic O₂ gradients were explicitly demonstrated to play a role in the induction of zonal CYP2B distributions.

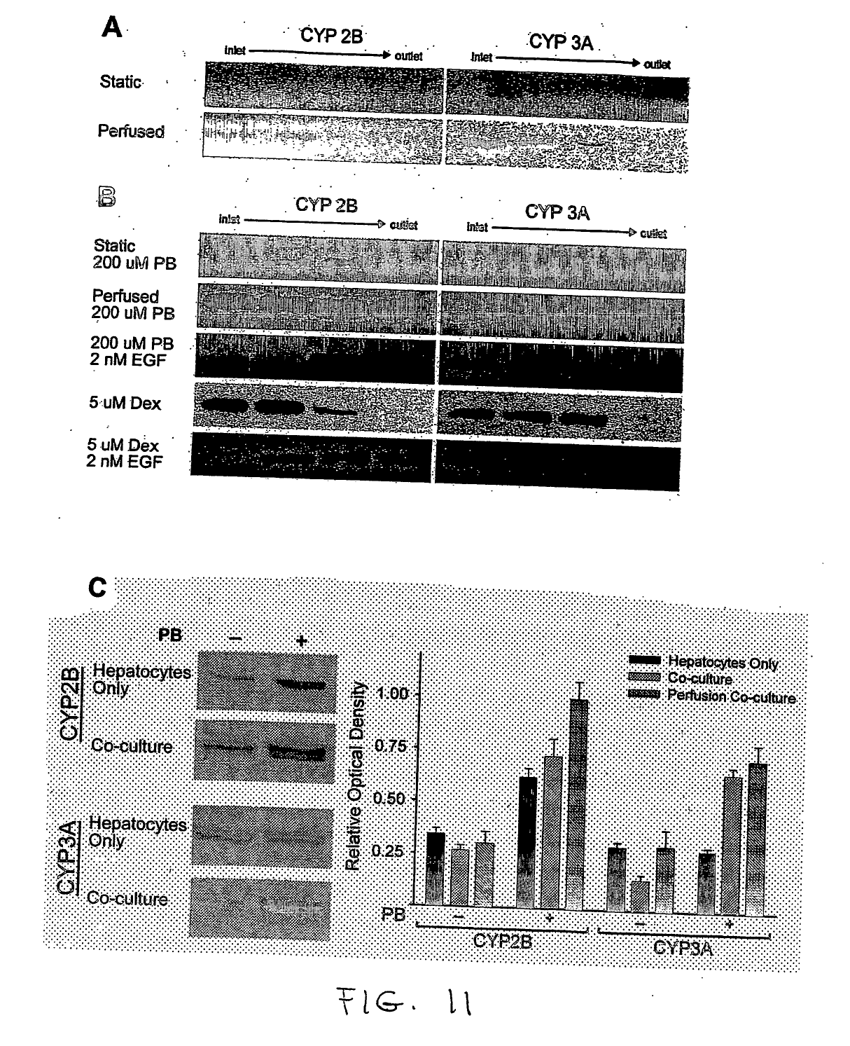

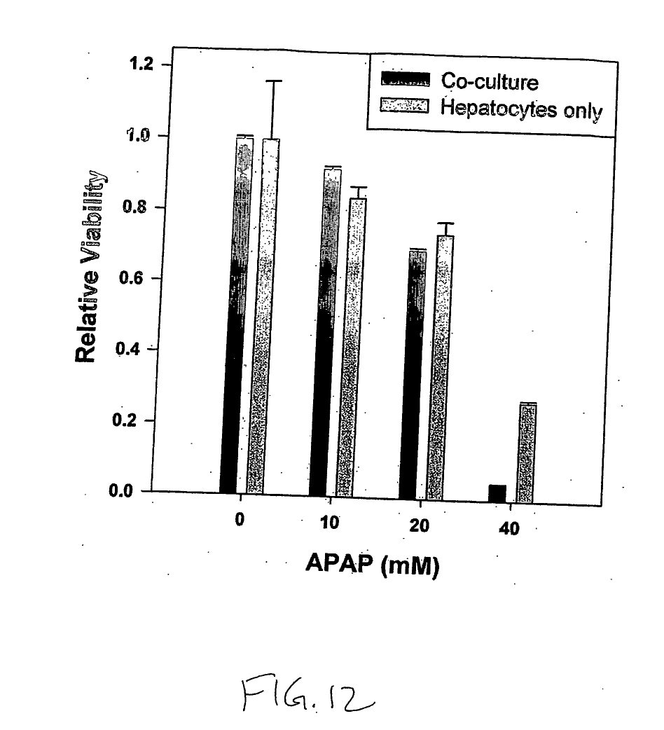

Furthermore, without induction, both CYP2B and CYP3A protein was present at low levels after 48 hour perfusion with little distinguishable spatial heterogeneity as compared to not detectable protein under static culture conditions. (FIG. 11). Next, induction of static cultures with phenobarbital (PB) over the same time period resulted in moderate CYP2B expression and low CYP3A (FIG. 12). Dramatic expression of both CYPs over controls was seen after only 36 hours when cultures were perfused with PB. Though expression of CYP2B was increased in all regions, levels were highest in the lower-oxygen outlet regions. Similarly, CYP3A protein showed increasing expression from inlet to outlet. Based on previous studies that showed repression of PB-induced CYP2B expression by epidermal growth factor (EGF), added 2 nM EGF to the perfusion media. At a dose of 200 μM PB, EGF did not significantly alter CYP2B levels along the length of the chamber though maximal levels were noted in the outlet regions. CYP3A levels in response to PB and EGF also showed little difference from PB-only perfusion displaying maximal expression at the outlet.

Experiments were also carried out to evaluate dexamethasone (DEX) as an inducer of CYPs in this perfusion system. DEX induced CYP2B to high levels which were localized to inlet regions of the culture. For CYP3A, induction was mostly uniform, but not detectable in the outlet region. When EGF was added to DEX-perfused cultures, a significant shift in CYP2B spatial distribution was noted from inlet regions to the outlet. CYP3A induction remained uniform in response to DEX and EGF, but was extend across all regions of the culture.

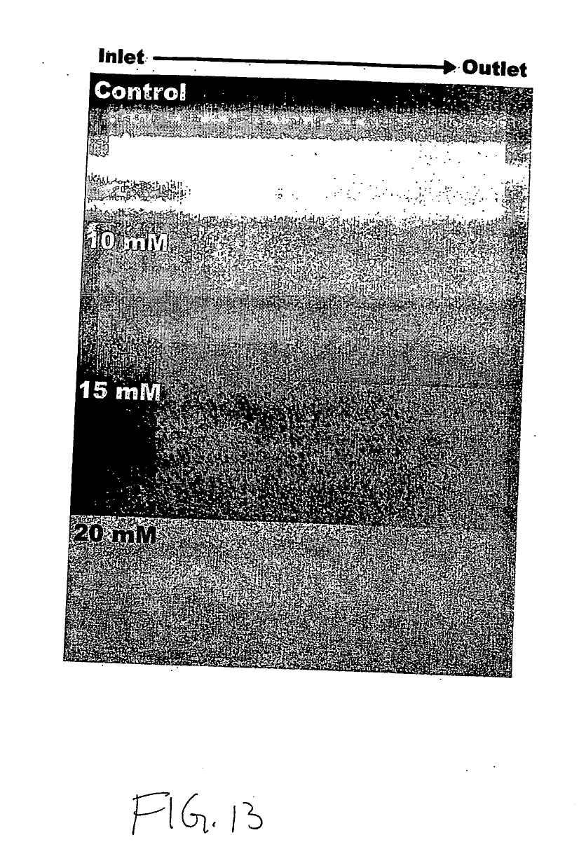

Acetaminophen (APAP) was evaluated for its acute toxic effect on hepatocyte cultures and co-cultures (Figure Static toxicity dose response of APAP). Viability, as assessed by MTT, decreased in a dose-dependant manner with reduced viability of 5% in hepatocytes alone and 28% in co-culture at 40 mM APAP after 24 hours. These data suggested that a dose range from 0-20 mM APAP would result in moderate toxicity in bioreactor cultures. FIG. 13 shows a panel of images of the full length (˜5.6 cm) of the bioreactor cultures perfused with various concentrations of APAP for 24 hours and then incubated with MTT. The presence and intensity of purple precipitate is proportional to cell viability. Of note is the dramatic decrease in staining from the inlet to the outlet region at a dose of 15 mM APAP as compared to control (moderate decrease) and 20 mM (no staining).

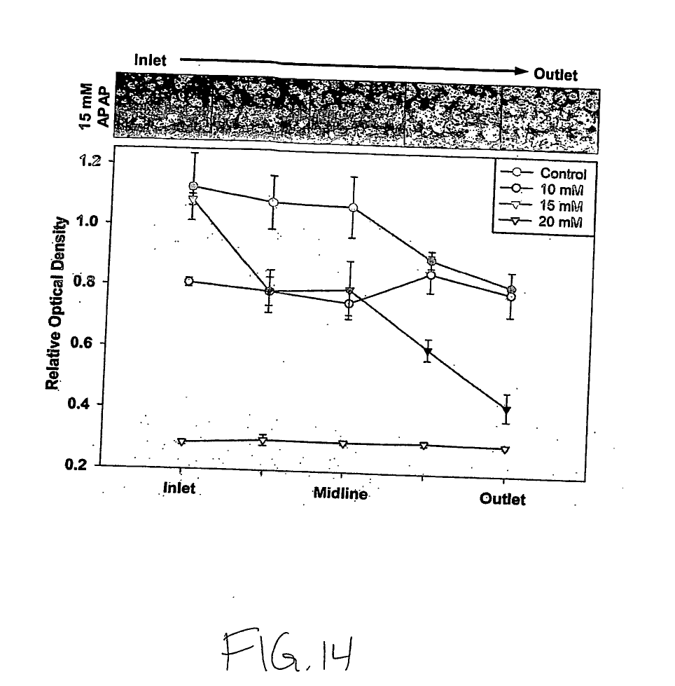

For further quantification of regional variations in viability, bright-field images were acquired at low magnification (40×) along the length of the culture for measurement of mean optical density (FIG. 14). Under the control condition, viability decreased 30% from inlet to outlet. However, at 10 mM APAP, toxicity was more uniform over the culture but was decreased to 80% of average control viability. Administration of 15 mM APAP resulted in maximal toxicity in the outlet region, decreased 70% from the inlet region. At the highest dose, 20 mM, toxicity was virtually complete.

Many members of the CYP superfamily responsible for phase I drug and steroid biotransformation are expressed in a zonal pattern in vivo. Among the determinants of the pericentral localization of CYPs under both normal and induced conditions are gradients of oxygen, nutrients, and hormones. Recapitulation of these dynamic gradients in bioreactor cultures resulted in spatial distributions of both CYP2B and CYP3A that mimic those found in vivo. Additionally, CYP induction was potentiated by the perfusion microenvironment of the reactor as shown by the dramatic increase in protein levels over static cultures in response to 200 μM PB. Previous studies demonstrated that the repressive effects of EGF on PB induction are modulated by oxygen.

Addition of EGF with PB in the current study did not significantly alter the spatial CYP2B pattern, but in conjunction with DEX, EGF shifted maximal CYP2B expression from the inlet to the outlet. This shifting effect, also noted to a lesser extent in CYP3A expression, may be due the formation of EGF gradients, thus demonstrating how dynamic gradients of growth factors and hormones regulate CYP zonation. Finally, though overlapping CYP2B and CYP3A induction by PB and DEX is likely mediated by nuclear hormone receptors such as constitutive androstane and pregnane X, the mechanism by which oxygen availability and hormones can modulate these pathways remains to be elucidated.

The proposed mechanism of APAP hepatotoxicity involves the formation of a reactive intermediate, NAPQI, which initiates free-radial damage of intracellular structures. Toxic effects in this study are likely due to the depletion of glutathione, which provides protective inactivation of NAPQI. Though pericentral localization of APAP toxicity in vivo has been attributed to local expression of CYP isoenzymes 2E1 and 3A, reduced oxygen availability in centrilobular regions may also contribute by depleting ATP and glutathione, or increasing damage by reactive species. A combination of these factors likely resulted in the regional toxicity observed in reactor cultures under dynamic oxygen gradients. Demonstration of zonal toxicity in vitro allows decoupling of the effects of CYP bioactivation and glutathione levels on acute APAP toxicity.

Furthermore, this system may allow elucidation of the actions various clinically important compounds such as ethanol or N-acetyl-cysteine and their respective exacerbating or protective effects on APAP toxicity.

As demonstrated by the data, oxygen gradients were applied to cultures of rat hepatocytes to develop and in vitro model of liver zonation. Provided is a model of oxygen transport considering both analytical and numerical solutions to the governing equation (Eq. (1)) derived from species continuity assumptions. Cells experienced oxygen conditions ranging from normoxia to hypoxia without compromising viability as shown by morphology and fluorescent markers of membrane integrity (FIG. 9). The hepatocytes exposed to oxygen gradients exhibited characteristics of in vivo zonation upon induction as shown by PEPCK (predominantly upstream) and CYP2B (predominantly downstream) protein levels. With this in vitro model of liver zonation, the microenvironmental conditions seen in the liver sinusoid that are thought to be responsible for heterogeneous distribution of metabolic and detoxifying functions can be reproduced.

Perfusion bioreactor systems, particularly those containing hepatocytes, are typically evaluated with respect to design criteria such as reactor geometry, flow parameters, and nutrient transport. The bioreactor provided by the disclosure offers a simple Cartesian geometry that provides a uniform flow field in which transport phenomena can be easily modeled. Small-scale experimental reactors have an additional advantage of allowing in situ analysis of cellular responses at the molecular level as well as bulk functional assays.

Cell seeding conditions and cell height should be kept uniform within the bioreactor system to insure uniformity of the flow field. The bioreactor experiments carried out in the specific examples herein, were typically conducted at a flow rate of 0.5 mL/min, corresponding to a shear stress of 1.25 dyne/cm², although higher stress near 7.5 dynes/cm² may have been present at higher flow rates using validation experiments.

Oxygen measurements were taken over a wide range of flow conditions with two different inlet pO₂ levels (FIG. 7) to show that oxygen gradients could be predicted and controlled to achieve a desired profile. Both analytical and numerical solutions were evaluated for purposes of comparing assumptions about oxygen uptake rate in hepatocytes. The analytical solution (Eq. (7)) to the model overestimated oxygen consumption in low oxygen environments due to the assumption of constant oxygen uptake. A numerical solution that incorporated an explicit approximation of Michaelis-Menten oxygen consumption kinetics more closely correlated with measured values. Though oxygen uptake in rat hepatocytes has been reported to decline after isolation in monolayer culture, our model did not take into account these changes and thus assumed constant Vₘₐₓ, and Kₙ values (Table 1) that have been reported for day 1 post-isolation. The experiments showed that physiological oxygen gradients (76 mmHg pO₂ inlet) provided more favorable and reproducible operation due the sensitivity of the gradient slope to small changes in flow rate with higher inlet conditions (158 mmHg pO₂ inlet). The advantage of implementing physiological gradients was seen in operational optimization as well as in the induction of zonal functions modulated by oxygen.

Though PEPCK activation occurs mainly via a cAMP secondary signal to glucagon binding, the mechanisms by which oxygen can modulate activation is still being elucidated. In the in vitro system, rat hepatocytes, when exposed to a continuous range of oxygen concentrations, could exhibit a heterogeneous distribution of PEPCK that correlates with periportal and perivenous localization seen in vivo. Control experiments showed that in the absence of a physiologic oxygen gradient, glucagon dependent PEPCK activation was uniform along the length of the reactor chamber.

Several cytochrome P450 isoenzymes have been localized to perivenous regions of the liver. The induction of CYP2B by phenobarbital has also been shown to be modulated by EGF and oxygen. Previous studies indicate that EGF repression of the PB-dependent induction of CYP2B is lost under perivenous pO₂, resulting in zonal expression pattern that correlates with the in vivo distribution. Consistent with this finding, the in vitro system provided by the disclosure showed increasing CYP2B induction along the length of the chamber when exposed to PB and EGF, with maximal induction in the low-oxygen perivenous-like region. Under the given operating conditions, an EGF gradient may be contributing to the zonal pattern of CYP2B, in as much as repression of PB-dependent CYP2B activation would be strong in the inlet region and weak at the EGF-depleted outlet region. However, imposing a supraphysiologic oxygen gradient with the same EGF profile did not result in significant differences in CYP2B levels from inlet to outlet, indicated that oxygen was primarily responsible for heterogeneous CYP2B distribution. Further studies examining zonal detoxification could use this same methodology to induce heterogeneous distributions of other P450 isoenzymes such as CYP3A4 or CYP2EI. In addition, with tight control of flow parameters, the kinetics of zonal induction, both for metabolic and detoxification processes, could be examined by retrograde perfusion methods.

The data provided herein support the observations that oxygen is an important modulator of cell function. In the case of hypoxia-dependent changes in gene expression in which the heterodimeric transcription factor HIF-1I plays a major role, heme proteins have been suggested as the purported oxygen sensor. Though no ubiquitous heme molecule has been identified as an oxygen sensor, observations that transition metals (Co, Ni, and Mn) and iron chelators induce HIF-1I while competitive heme binding by CO or NO reduces HIF-1I activity support this hypothesis. In addition, hydrogen peroxide may act a second messenger downstream of a heme binding event to modulate transcription factor binding. Exogenously added H₂O₂ in hepatocyte cultures paralleled the effect of periportal oxygen by enhancing the glucagon-dependent induction of PEPCK while Hela cells H₂O₂ resulted in destabilization of HIF-1I. The heme-based O₂ sensing model is consistent with the modulation of the PEPCK via a normoxia response element, but HIF-1I has not yet been implicated in the oxygen-dependent regulation of CYP2B expression, suggesting a more direct role of heme proteins in CYP gene expression. Independent of the mechanism, however, the gradient system presented here can provide a continuous range of oxygen tensions in which the functional range of candidate oxygen sensors may be determined.

The disclosure provides a bioreactor that allows steady-state oxygen gradients to be imposed upon in vitro culture systems. The bioreactor system of the disclosure has been applied to liver zonation and have shown that physiological oxygen gradients contribute to heterogeneous induction of PEPCK and CYP2B that mimics distributions in vivo. The results demonstrate the ability of oxygen to modulating gene expression and imply that oxygen plays an important role in the maintenance of liver-specific metabolism in a bioreactor system. In addition, considerations of the effect of oxygen gradients in the design and optimization current bioartificial support systems may serve to improve their function. Other applications of the gradient system might involve examination of ischemia-reperfusion injury, the mechanisms of ischemic preconditioning being attempted in organ preservation, and mechanisms of zonal toxicity such as that caused by carbon tetrachloride or acetaminophen. This approach is generally applicable to systems that can benefit from (i) a continuous range of O₂ concentration; (ii) dynamics; (iii) large cell populations for molecular characterization; and (iv) the role flow and soluble factors on cell function.

A number of embodiments of the invention have been described. Nevertheless, it will be understood that various modifications may be made without departing from the spirit and scope of the invention. Accordingly, other embodiments are within the scope of the following claims.

What is claimed is:

1. A method comprising:
controlling an oxygen gradient across a population of cells in one or more bioreactors to modify a tissue morphology, function, and/or gene expression.

2. The method of claim 1, wherein the population of cells are substantially homogenous.

3. The method of claim 1, wherein the population of cells comprise two or more cell types.

4. The method of claim 3, wherein the two or more cell types comprise stromal cells and a cell type selected from the group consisting of hepatocytes, pancreatic cells, endothelial cells, epithelial cells, cancer cells, muscle cells, and kidney cells.

5. The method of claim 1, wherein the population of cells are selected from the group consisting of hepatocytes, pancreatic cells, endothelial cells, epithelial cells, cancer cells, muscle cells, kidney cells, and stromal cells.

6. The method of claim 1, wherein the population of cells comprises hepatocytes.

7. The method of claim 1, wherein the population of cells comprises stromal cells.

8. The method of claim 1, wherein the population of cells comprises a co-culture of stromal cells and hepatocytes.

9. The method of claim 8, wherein the co culture of stromal cells and hepatocytes are in a micropattern formation.

10. The method of claim 1, wherein the one or more bioreactors comprise:
a pump,
a gas exchange device,
at least one culture device comprising,
at least one housing;
at least one substrate,
at least one tissue binding surface on each of the at least one substrate,
wherein the housing comprises at least one wall, an inlet port and an outlet port, wherein the housing fluidly seals the tissue binding surface to provide a flow space in fluid communication with the inlet and outlet ports,
a gas sensor, and
a fluid reservoir,
wherein the pump, the gas exchange device, the culture device, the gas sensor and the fluid reservoir are in fluid communication, such that a fluid is pumped from the fluid reservoir through (i) the gas exchanger, (ii) the culture device, (iii) the gas sensor and returned to the fluid reservoir using the pump and wherein the population of cells is cultured on the tissue binding surface of the substrate and wherein the gas concentration is modulated by the gas exchange device and sensed by the gas sensor.

11. The method of claim 10, wherein the bioreactor further comprises a bubble trap between the gas exchange device and the culture device.

12. The method of claim 10, wherein the gas exchange device modifies the O₂ content of the fluid.

13. The method of claim 12, wherein the O₂ content is higher proximal to the inlet port of the culture device and decreases further distal from the inlet port.

14. The method of claim 10, wherein the gas exchange device comprises a gas sensor.

15. The method of claim 10, wherein the fluid is growth medium.

16. The method of claim 10, wherein the pump is a peristaltic pump.

17. The method of claim 10, wherein the pump is a syringe pump.

18. The method of claim 10, wherein the substrate is biocompatible.

19. The method of claim 18, wherein the tissue binding surface of the substrate comprises a material selected from the group consisting of polyamides; polyesters; polystyrene; polypropylene; polyacrylates; polyvinyl compounds; polycarbonate (PVC); polytetrafluoroethylene (PTFE); nitrocellulose; cotton; polyglycolic acid (PGA); cat gut sutures; cellulose; dextran; gelatin; and glass.

20. The method of claim 18, wherein the substrate is modified to promote cell adhesion.

21. A bioreactor comprising:
at least one housing having an inlet port and an outlet port;
at least one substrate disposed in the at least one housing;
at least one tissue binding surface on each of the at least one substrate, the housing and tissue binding surface defining a flow space along the tissue binding surface;
a pump in fluid communication with the inlet port and the outlet port of the housing;
a gas exchange device disposed between the pump and the inlet port;
a fluid reservoir in fluid communication with the pump; and
a gas sensor disposed between the outlet port and the fluid reservoir,
wherein the pump, the gas exchange device, the flow space, the gas sensor and the fluid reservoir are in fluid communication, such that a fluid is pumped from the fluid reservoir through (i) the gas exchanger, (ii) the flow space, (iii) the gas sensor and returned to the fluid reservoir using the pump and wherein the gas concentration is modulated by the gas exchange device and sensed by the gas sensor.

22. The bioreactor of claim 21, further comprising a tissue disposed on the tissue binding surface.

23. The bioreactor of claim 22, wherein the tissue comprises parenchymal cells.

24. The bioreactor of claim 22, wherein the tissue comprises stromal cells.

25. The bioreactor of claim 23, wherein the tissue further comprises stromal cells.

26. The bioreactor of claim 23, wherein the parenchymal cells are hepatocyte cells.

27. The bioreactor of claim 25, wherein the parenchymal cells are hepatocyte cells.

28. The bioreactor of claim 21, wherein the substrate is substantially planar.

29. The bioreactor of claim 21, wherein the substrate is concave or convex.

30. The bioreactor of claim 21, wherein the at least one substrate comprises a plurality of substrates.

31. The bioreactor of claim 21, wherein the bioreactor further comprises a bubble trap between the gas exchange device and the inlet port.

32. The bioreactor of claim 21, wherein the gas exchange device modifies the O₂ content of the fluid.

33. The bioreactor of claim 32, wherein the O₂ content is higher proximal to the inlet port of the housing and decreases further distal from the inlet port.

34. The bioreactor of claim 21, wherein the gas exchange device comprises a gas sensor.

35. The bioreactor of claim 21, wherein the fluid is growth medium.

36. The bioreactor of claim 21, wherein the pump is a peristaltic pump.

37. The bioreactor of claim 21, wherein the pump is a syringe pump.

38. The bioreactor of claim 21, wherein the substrate is biocompatible.

39. The bioreactor of claim 21, wherein the tissue binding surface of the substrate comprises a material selected from the group consisting of polyamides; polyesters; polystyrene; polypropylene; polyacrylates; polyvinyl compounds; polycarbonate (PVC); polytetrafluoroethylene (PTFE); nitrocellulose; cotton; polyglycolic acid (PGA); cat gut sutures; cellulose; dextran; gelatin; and glass.

40. The bioreactor of claim 21, wherein the substrate is modified to promote cell adhesion.

41. The bioreactor of claim 21, comprising one substrate and a plurality of tissue binding surface on the at least one substrate.

42. A method of producing a tissue, comprising:
seeding a population of cells on a substrate in a bioreactor system;
controlling an oxygen gradient across the population of cells in one or more bioreactors;
culturing the cells under conditions and for a sufficient period of time to generate a tissue.

43. The method of claim 42, wherein the population of cells are substantially homogenous.

44. The method of claim 42, wherein the population of cells comprise two or more cell types.

45. The method of claim 44, wherein the two or more cell types comprise stromal cells and a cell type selected from the group consisting of hepatocytes, pancreatic cells, endothelial cells, epithelial cells, cancer cells, muscle cells, and kidney cells.

46. The method of claim 42, wherein the population of cells are selected from the group consisting of hepatocytes, pancreatic cells, endothelial cells, epithelial cells, cancer cells, muscle cells, kidney cells, and stromal cells.

47. The method of claim 42, wherein the population of cells comprises hepatocytes.

48. The method of claim 42, wherein the population of cells comprises stromal cells.

49. The method of claim 42, wherein the population of cells comprises a co-culture of stromal cells and hepatocytes.

50. The method of claim 49, wherein the co-culture of stromal cells and hepatocytes are in a micropattern formation.

51. The method of claim 42, wherein the bioreactor comprises:
a pump,
a gas exchange device,
at least one culture device comprising,
at least one housing;
at least one substrate,
at least one tissue binding surface on each of the at least one substrate,
wherein the housing comprises at least one wall, an inlet port and an outlet port, wherein the housing fluidly seals the tissue binding surface to provide a flow space in fluid communication with the inlet and outlet ports,
a gas sensor, and
a fluid reservoir,
wherein the pump, the gas exchange device, the culture device, the gas sensor and the fluid reservoir are in fluid communication, such that a fluid is pumped from the fluid reservoir through (i) the gas exchanger, (ii) the culture device, (iii) the gas sensor and returned to the fluid reservoir using the pump, wherein the population of cells is cultured on the tissue binding surface of the substrate and wherein the gas concentration is modulated by the gas exchange device and sensed by the gas sensor.

52. The method of claim 51, wherein the bioreactor further comprises a bubble trap between the gas exchange device and the culture device.

53. The method of claim 51, wherein the gas exchange device modifies the O₂ content of the fluid.

54. The method of claim 53, wherein the O₂ content is higher proximal to the inlet port of the culture device and decreases further distal from the inlet port.

55. The method of claim 51, wherein the gas exchange device comprises a gas sensor.

56. The method of claim 51, wherein the fluid is growth medium.

57. The method of claim 51, wherein the pump is a peristaltic pump.

58. The method of claim 51, wherein the pump is a syringe pump.

59. The method of claim 51, wherein the substrate is biocompatible.

60. The method of claim 59, wherein the tissue binding surface of the substrate comprises a material selected from the group consisting of polyamides; polyesters; polystyrene; polypropylene; polyacrylates; polyvinyl compounds; polycarbonate (PVC); polytetrafluoroethylene (PTFE); nitrocellulose; cotton; polyglycolic acid (PGA); cat gut sutures; cellulose; dextran; gelatin; and glass.

61. The method of claim 59, wherein the substrate is modified to promote cell adhesion.

62. A tissue produced by the method of claim 42.

63. An assay system comprising:
contacting a tissue produced by the method of claim 42 with a test agent and measuring an activity selected from gene expression, cell function, metabolic activity, morphology, and a combination thereof, of the tissue.

64. The assay system of claim 63, wherein the test agent is selected from a protein, a peptide, a polypeptide, an antibody, a peptidomimetic, a small molecule, an oligonucleotide, and a polynucleotide.

65. The assay system of claim 63, wherein the test agent is a cytotoxic agent.

66. The assay system of claim 63, wherein the test agent is a pharmaceutical agent.

67. The assay system of claim 63, wherein the test agent is a xenobiotic.

68. The assay system of claim 67, wherein the xenobiotic is selected from the group consisting of an environmental toxins, chemical/biological warfare agents, natural compounds such as holistic therapies and nutraceuticals.

69. The assay system of claim 63, wherein the activity is adsorption, distributions, metabolism, excretion, and toxicology (ADMET) of the test agent.

70. The assay system of claim 63, wherein the metabolic activity is protein production.

71. The assay system of claim 63, wherein the metabolic activity is enzyme bioproduct formation.
